SEQUENCE LISTING (2 sequences)

SEQ ID NO: 1 - DNA, 34 bp, Artificial Sequence
  Artificially synthesized nucleotide sequence
  misc_feature LOCATION: (33)..(34): n is a, c, g, or t
tttttttaagcagtggtatcaacgcagagtacnn

SEQ ID NO: 2 - DNA, 30 bp, Artificial Sequence
  Artificially synthesized nucleotide sequence
tttttttttttttttttttttttttttttt

(19) United States
(12) Patent Application Publication
(10) Pub. No.: US 2023/0212556 A1
(43) Pub. Date: Jul. 6, 2023

(54) SYSTEMS AND METHODS FOR ASSOCIATING SINGLE CELL IMAGING WITH RNA TRANSCRIPTOMICS

(71) Applicants: Cell Microsystems, Inc., Durham, NC (US); The Trustees of Columbia University in the City of New York, New York, NY (US)

(72) Inventors: Jacquelyn DuVall, Durham, NC (US); Brandon Thompson, Durham, NC (US); Peter Alan Sims, New York, NY (US); Jinzhou Yuan, Edgewater, NJ (US); Zhouzerui Liu, New York, NY (US); Dogukan Mizrak, Ann Arbor, MI (US); Steven C. Gebhart, Durham, NC (US); Peter Glyn Boone, Durham, NC (US)

(21) Appl. No.: 17/622,527

(22) PCT Filed: Jun. 26, 2020
(86) PCT No.: PCT/US2020/039943

§ 371 Date: Dec. 23, 2021

Related U.S. Application Data
(60) Provisional application No. 62/867,830, filed on Jun. 27, 2019.

(51) Int. Cl. C12N 15/10; C12Q 1/6813; C12Q 1/6874; C12M 1/36

(52) U.S. Cl. CPC C12N 15/1065; C12M 41/48; C12N 15/1093; C12N 15/1096; C12Q 1/6813; C12Q 1/6874

(57) ABSTRACT

Systems and methods for associating single cell imaging data with RNA transcriptomics. Single cells are isolated into microwells with a microbead having oligonucleotides conjugated on its surface. Each oligonucleotide includes a cell identifying optical barcode that is unique to that bead and binding sequence for RNA capture after cell lysis. The system is configured for loading single cells into the microarray and for flowing cell lysis buffers and other reagents into the microarray for performing RNA library sample preparation. The system is also configured for lowing optical hybridization probes that are complementary to the cell identifying optical barcodes and optically labeled onto the microwell array and for obtaining images of the microwells in response to the probes. The system and unique cell identifying optical barcodes and complementary optical hybridization probes facilitate a link between phenotypic imaging of cells resident on the microwell array with single cell whole transcriptome sequencing.

### CROSS-REFERENCE TO RELATED APPLICATIONS

This application is a 35 U.S.C. Section 371 national phase application of PCT International Patent Application No. PCT/US2020/039943, filed Jun. 26, 2020, incorporated herein by reference in its entirety and which claims the benefit of U.S. Provisional Pat. Application Serial No. 62/867,830, filed Jun. 27, 2019, the disclosure of which is incorporated herein by reference in its entirety.

### STATEMENT REGARDING FEDERALLY SPONSORED RESEARCH

This invention was made with government support under Grant Nos. 9R44HG010003-02A1, 5R44HG010003-03, 75N91019C00029, CA202827, and HG010003 awarded by the National Institutes of Health. The government has certain rights in the invention.

### TECHNICAL FIELD

This specification relates generally to automated systems and methods for associating single cell imaging with whole genome RNA transcription profiling.

### BACKGROUND

Recent advances in microfluidics and cDNA barcoding have led to a dramatic increase in the throughput of single-cell RNA-Seq (scRNA-seq)[1-5]. However, unlike earlier or less scalable techniques[6-8], these new tools do not offer a straightforward way to directly link phenotypic information obtained from individual, live cells to their expression profiles. Nonetheless, microwell-based implementations of scRNA-seq are compatible with a wide variety of phenotypic measurements including live cell imaging, immunofluorescence, and protein secretion assays[3, 9-12]. These methods involve co-encapsulation of individual cells and barcoded RNA capture beads in arrays of microfabricated chambers. Because the barcoded beads are randomly distributed into microwells, one cannot directly link phenotypes measured in the microwells to their corresponding expression profiles.

The present disclosure provides automated systems and methods for associating single cell imaging data with whole genome RNA transcription profiling.

### SUMMARY

This specification describes methods and systems for automated single cell imaging and sample preparation that enable association of single cell imaging data with RNA transcriptomics. An example system includes an instrument assembly comprising a fluidics subsystem, a thermal subsystem, and an imaging subsystem including a motorized stage configured for holding and scanning a microwell array. The system includes a control subsystem coupled to the instrument assembly, and the control subsystem is configured for performing operations. The operations include flowing, using the fluidics subsystem, a plurality of cells onto the microwell array, wherein a subset of the cells reside as single cells in the microwells and obtaining, for each position in the microwell array, one or more first images at the position using the imaging subsystem. The control subsystem is configured for flowing, using the fluidics subsystem, microbeads having a cell identifying optical barcode sequence and an RNA binding sequence onto the microwell array, wherein a subset of the beads reside as a single cell-bead pair in the microwells. The control subsystem is configured for flowing, using the fluidics subsystem, a cell lysis buffer and one or more reagents for RNA library preparation onto the microwell array. The control subsystem is configured for flowing, using the fluidics subsystem, a first of N pools of a plurality of optical hybridization probes onto the microwell array and hybridizing the probes to the beads located therein having a complementary nucleotide sequence in the cell identifying optical barcode sequence attached thereto. The control subsystem is configured for obtaining, for each position, one or more second images to quantify a fluorescent intensity at the position using the imaging subsystem, each of the one or more second images used to create a binary code depicting a match or a lack of a match between at least one of the optical hybridization probes and the cell identifying optical barcodes. The control subsystem is configured for repeating the flowing and hybridizing step and obtaining of the one or more second images step for each of the N pools of probes. The control subsystem is configured for determining, by mapping the binary code for each of the N pools of probes to the cell identifying barcode sequence, for each position the cell identifying optical barcode for the position using the second images and storing a data association between the cell identifying optical barcode for the position and the first image at the position.

An example method includes an automated method for associating single cell imaging data with RNA transcriptomics. The method includes flowing, using a fluidics subsystem, a plurality of cells onto a microwell array, wherein a subset of the cells reside as single cells in the microwells; obtaining, for each position of a plurality of positions in a microwell array, one or more first images at the position using an imaging subsystem; flowing, using the fluidics subsystem, a plurality of microbeads having a cell identifying optical barcode sequence and an RNA binding sequence onto the microwell array, wherein a subset of the beads reside as a single cell-bead pair in the microwells; flowing, using the fluidics subsystem, a cell lysis buffer and one or more reagents for RNA library preparation onto the microwell array; flowing, using the fluidics subsystem, a first of N pools of a plurality of optical hybridization probes onto the microwell array and hybridizing the probes to the beads located therein having a complementary nucleotide sequence in the cell identifying optical barcode sequence attached thereto. The control subsystem is configured for obtaining, for each position of the plurality of positions, one or more second images to quantify a fluorescent intensity at the position using the imaging subsystem, each of the one or more second images used to create a binary code depicting a match or a lack of a match between at least one of the optical hybridization probes and the cell identifying optical barcodes; repeating the flowing and hybridizing step and obtaining of the one or more second images step for each of the N pools of probes; determining, by mapping the binary code for each of the N pools of probes to the cell identifying barcode sequence, for each position of the plurality of positions, the cell identifying optical barcode for the position using the second images and storing a data association between the cell identifying optical barcode for the position and the first image at the position; and storing, for each position of the plurality of positions, after receiving nucleic acid sequencing data for each cell identifying optical barcode, a data association between the nucleic acid sequencing data, the cell identifying optical barcode, and the first image associated with the cell identifying optical barcode.

The computer systems described in this specification may be implemented in hardware, software, firmware, or any combination thereof. In some examples, the computer systems may be implemented using a computer readable medium having stored thereon computer executable instructions that when executed by the processor of a computer control the computer to perform steps. Examples of suitable computer readable media include non-transitory computer readable media, such as disk memory devices, chip memory devices, programmable logic devices, and application specific integrated circuits. In addition, a computer readable medium that implements the subject matter described herein may be located on a single device or computing platform or may be distributed across multiple devices or computing platforms.

An example method is provided for identifying a correspondence between single cell optical phenotypes and cell type, lineage, or clone. The method includes: initializing a system, the system comprising: an instrument assembly comprising a fluidics subsystem, a thermal subsystem, and an imaging subsystem, wherein the imaging subsystem comprises a stage configured for holding a microwell array; a control subsystem coupled to the instrument assembly, the control subsystem comprising at least one processor and memory; and using the control subsystem for performing operations. The operations including flowing, using the fluidics subsystem, a plurality of cells onto the microwell array, wherein a subset of the cells reside as single cells in the microwells; obtaining, for each position of a plurality of positions in the microwell array, one or more first images at the position using the imaging subsystem and measuring one or more of a cell optical phenotypic feature; flowing, using the fluidics subsystem, a plurality of microbeads having a cell identifying optical barcode sequence and an RNA binding sequence onto the microwell array, wherein a subset of the beads reside as a single cell-bead pair in the microwells; flowing, using the fluidics subsystem, a cell lysis buffer and one or more reagents for RNA library preparation onto the microwell array; flowing, using the fluidics subsystem, a first of N pools of a plurality of optical hybridization probes onto the microwell array and hybridizing the probes to the beads located therein having a complementary nucleotide sequence in the cell identifying optical barcode sequence; obtaining, for each position of the plurality of positions, one or more second images to quantify a fluorescent intensity at the position using the imaging subsystem, each of the one or more second images used to create a binary code depicting a match or a lack of a match between at least one of the optical hybridization probes and the cell identifying optical barcodes; repeating the flowing and hybridizing step and obtaining of the one or more second images step for each of the N pools of probes; determining, by mapping the binary code for each of the N pools of probes to the cell identifying barcode sequence, for each position of the plurality of positions, the cell identifying optical barcode for the position, and storing a data association between the cell identifying optical barcode for the position and the first image at the position; storing, for each position of the plurality of positions, after receiving nucleic acid sequencing data for each cell identifying optical barcode, a data association between the nucleic acid sequencing data, the cell identifying optical barcode, and the first image associated with the cell identifying optical barcode. The method includes generating a representation of the relationship between the one or more cell optical phenotypic features and the nucleic acid sequencing data associated with each of the first images, wherein a correlation between the single cell phenotypic features and the associated sequencing data identifies a correspondence between single cell optical phenotypes and cell type, lineage, or clone based on transcriptomics of that single cell.

The automated system and methods of the present disclosure can be used for preparation of nucleic acid sequencing libraries in addition to preparation of RNA libraries. For example, a plurality of microbeads having a cell identifying optical barcode sequence and a primer sequence for capture of cellular nucleic acid can be flowed onto the microwell array. The primer sequence can be an oligo(dT) to capture RNA, mRNA, and non-coding RNA; a random sequence to capture any DNA or RNA; or a specific sequence targeted to a DNA loci or an RNA transcript. In this manner the automated system is provided for associating single cell imaging with unique optical barcode readout, and preparation of nucleic acid libraries. Similarly, an automated method is provided for associating single cell imaging data with nucleic acid sequencing data. In addition, a method for identifying a correspondence between single cell optical phenotypes and cell type, lineage, or clone is provided, where a correlation between the single cell phenotypic features and the associated sequencing data identifies a correspondence between single cell optical phenotypes and cell type, lineage, or clone based on nucleic acid sequence of that single cell.

### BRIEF DESCRIPTION OF THE DRAWINGS

FIGS. 1A-1C are diagrams of an example automated system for associating a single cell image with unique optical barcode readout, and preparation of RNA libraries;

FIG. 2 illustrates an example mechanical device for implementing the system;

FIG. 3A shows a 3D model of the device with enclosure;

FIG. 3B shows an example implementation of the device with a side cover removed to illustrate internal components;

FIG. 4 shows a 3D model of an example imaging subsystem;

FIG. 5A is a top-down view of an example thermal subsystem;

FIG. 5B is a block diagram of an example subsystem including an interface between the reagent cartridge and the fluidic manifold;

FIGS. 6A-6B are flow diagrams of an example method for associating a single cell image with unique optical barcode readout, and preparation of RNA libraries using the automated system, to associate cell phenotypic data with whole genome RNA transcription sequence data;

FIGS. 6C-6F illustrate processes that may be carried out by the system;

FIGS. 7A-7C are schematic diagrams illustrating examples of the design of the microbeads having a plurality of attached oligonucleotides that include a PCR handle, a cell identifying optical barcode, a unique molecular identifier, and an oligo(dT) RNA binding sequence (A-top) and showing two different examples of a complementary optical hybridization probe hybridized to the cell identifying optical barcode (B-middle and C-bottom);

FIG. 8A displays data and images for the automated system of the present disclosure including cell loading (~1000 cells in a 10,000 microwell array), bright-field detection and fluorescence imaging of the loaded cells, bead loading (~8,500 beads in the 10,000 microwell array), and then cell lysis within the individual microwells of the array;

FIG. 8B displays images of cell lysis within the individual microwells of the array followed by a wash which shows removal of fluorescent cell lysate followed by graphs showing capillary and gel electrophoresis analysis of the bead-free PCR product extracted from beads subjected to the on-device workflow and negative control beads (beads that were not subjected to on-device workflow);

FIG. 9 is a flow diagram of an example method for automated cell imaging and sample preparation;

FIG. 10A shows a binary image of segmented and labeled microwells according to one or more embodiments of the present disclosure;

FIG. 10B shows a bright-field image of cells in microwells according to one or more embodiments of the present disclosure;

FIG. 10C shows a fluorescent image of live-stained cells according to one or more embodiments of the present disclosure;

FIG. 10D shows a fluorescent image of the microwells in FIG. 10C after cell lysis according to one or more embodiments of the present disclosure;

FIG. 11A is a schematic diagram illustrating an example of the design of the plurality of oligonucleotides attached to the microbeads that include a PCR handle, an 8-nucleotide unique molecular identifier (UMI) broken into 3 separate parts (NN, NN, and NNNN), a cell barcode S, a cell barcode Q, and an oligo(dT) RNA binding sequence in which the unique combination of the cell barcode S and cell barcode Q constitute the cell identifying optical barcode for each bead according to one or more embodiments of the presently disclosed subject matter;

FIG. 11B is a schematic diagram illustrating an example of split-pool, solid-phase synthesis of a set of microbeads with attached oligonucleotides including two 8-nucleotide sequences (cell barcode S and cell barcode Q, each of which is a member of a pool of 96 sequences), in which the unique combination of sequences after two rounds of split-pooling constitutes a total of 96² = 9,216 unique cell-identifying optical barcodes according to one or more embodiments of the presently disclosed subject matter;

FIG. 11C is a schematic diagram illustrating synthesis of sequential hybridization probe pools according to one or more embodiments of the presently disclosed subject matter;

FIG. 12 is a scatter plot showing the number of human- and mouse-aligning transcript molecules for each cell-identifying barcode in a single cell RNA-seq experiment performed using the automated system of the present disclosure illustrating that while the majority of cell-identifying barcodes are strongly associated with one species, some are associated with both, indicating co-encapsulation of multiple cells with a bead;

FIG. 13 shows violin plots of the distributions of the number of transcript molecules detected per cell for cell-identifying barcodes associated with either human or mouse transcriptome annotations (where at least 70% of molecules align to either the human or mouse transcriptome) from a single cell RNA-seq experiment using the automated system of the present disclosure;

FIG. 14 shows violin plots of the distributions of the number of genes detected per cell for cell-identifying barcodes associated with either human or mouse transcriptome annotations (where at least 70% of molecules align to either the human or mouse transcriptome) from a single cell RNA-seq experiment using the automated system of the present disclosure;

FIG. 15A shows images comparing raw and analyzed fluorescence images of 8-base, Cy3-labeled and 8-base, Cy5-labeled optical probes hybridized to the complementary cell identifying optical barcode on beads present in the individual microwells of the array in the automated system of the present disclosure;

FIG. 15B shows images of a cycle of fluorescence hybridization imaging in which a pooled set of 8-base, Cy5-labeled oligonucleotides and a set of 8-base, Cy3-labeled oligonucleotides were introduced into the array loaded with beads and imaged in each of channels 2 and 3 to probe the first and second sequences, respectively, on each bead in the automated system of the present disclosure;

FIG. 16 is an image showing software analysis of a cycle of fluorescence hybridization imaging to identify the two barcode sequences on each bead that together form the cell identifying optical barcode sequence. A pooled set of complementary optical probes consisting of 8-base, Cy5-labeled oligonucleotides and 8-base, Cy3-labeled oligonucleotides was introduced into the array device loaded with beads and imaged in each of channels 2 and 3, to probe the first and second barcode sequences, respectively, on each bead in the automated system of the present disclosure. The software analysis of this mix of pooled probes indicates the detected fluorescence as “positive” for channel 2, “positive” for channel 3, or positive for both;

FIG. 17A is a schematic diagram of a prophetic example illustrating optical decoding of cell identifying optical barcodes that can be performed in a ‘bead-by-bead’ decoding strategy. Scale bars: 50 µm (multi-well image) and 10 µm (single-well images) according to one or more embodiments of the present disclosure;

FIG. 17B is a bar plot of a prophetic example showing the fraction of scRNA-seq expression profiles that can be successfully linked to cell images in a comparison between ‘bead-by-bead’ and ‘cycle-by-cycle’ decoding methods according to one or more embodiments of the present disclosure;

FIG. 18A is a graph of a prophetic example showing molecular capture efficiency in violin plots showing the distribution of the number of molecules detectable per cell at different sequencing read depths in a mixed-species experiment according to one or more embodiments of the present disclosure;

FIG. 18B is a graph of a prophetic example showing molecular capture efficiency in violin plots showing the distribution of the number of genes detectable per cell at different sequencing read depths in a mixed-species experiment according to one or more embodiments of the present disclosure;

FIG. 18C is a scatter plot of a prophetic example showing linking accuracy that is obtainable according to one or more embodiments of the present disclosure by the number of uniquely aligned human and mouse reads of each cell identifying optical barcode that linked to images before removal of multiplets, as illustrated by the fluorescent intensity ratio of human and mouse live staining;

FIG. 18D is a scatter plot of a prophetic example showing linking accuracy that is obtainable according to one or more embodiments of the present disclosure by the number of uniquely aligned human and mouse reads of each cell identifying optical barcode that linked to images after removal of multiplets, as illustrated by the fluorescent intensity ratio of human and mouse live staining;

FIG. 19A shows paired optical and transcriptional phenotype measurement of cells in glioblastoma in a prophetic example plot of clustering of scRNA-seq expression profiles that shows the UMAP embedding of the cell score matrix from the single cell hierarchical Poisson factorization (scHPF) analysis of all linked cells in glioblastoma, according to one or more embodiments of the present disclosure;

FIG. 19B shows paired optical and transcriptional phenotype measurement of cells in glioblastoma in a prophetic example plot of clustering of scRNA-seq expression profiles that shows scores of cell lineage factors colored by the score of cell lineage factors from the scHPF analysis (the marker genes for each cell lineage factor are listed), according to one or more embodiments of the present disclosure;

FIG. 19C shows paired optical and transcriptional phenotype measurement of cells in glioblastoma in a prophetic example heatmap showing identification of imaging meta-features including the z-scored values of 16 cell imaging features, and a dendrogram showing three feature clusters, cell size, shape and Calcein staining intensity, from an unsupervised hierarchical clustering, according to one or more embodiments of the present disclosure;

FIG. 19D shows paired optical and transcriptional phenotype measurement of cells in glioblastoma in prophetic example boxplots illustrating clustering of scRNA-seq expression profiles that shows heterogeneity of cell imaging phenotypes and the distribution of imaging meta-features in each Phenograph cell cluster, according to one or more embodiments of the present disclosure;

FIG. 20 illustrates the relationships between optical phenotypes and transcriptional lineages in that the two major tumor cell lineages in glioblastoma can be distinguished just by clustering the imaging features as shown in a prophetic example plot of the two-dimensional diffusion map of malignant cells, colored by the cell imaging clusters, according to one or more embodiments of the present disclosure;

FIG. 21 includes a screenshot of an example GUI for controlling various aspects of the process;

FIG. 22 is another screenshot of an example GUI;

FIG. 23 is a screenshot of an example GUI for viewing live images of the microwell array in one of the fluorescence channels to set the imaging parameters for that channel of the scan;

FIG. 24 is a screenshot of an example GUI for setting various steps and their parameters for a cell loading operation;

FIG. 25 is a screenshot of an example GUI for viewing bright-field imaging results of a scan of the microwell array; and

FIG. 26 is a screenshot of another GUI for viewing fluorescence imaging results of a scan of the microwell array.

### DETAILED DESCRIPTION

Among the commercially available systems for single cell isolation and next generation sequencing (NGS) sample preparation, none are capable of associating a single cell image with a unique optical barcode readout, and preparation of single cell RNA libraries to enable association of single cell phenotypic data with RNA transcriptomics. This specification describes methods and systems which will allow high-quality multi-channel fluorescent imaging combined with automated single cell, whole transcriptome RNA library preparation, e.g., of several thousand single cells per 4-5 hour run. The system can establish single cell whole transcriptome sequencing (‘RNA-Seq’) data quality metrics. In operation, the system automates a capture of single cell images, association of a single cell image with a corresponding unique optical barcode readout (based on a unique cell identifying optical barcode sequence), and next generation sequencing (NGS) sample preparation method, referred to as Single Cell Optical Phenotyping and Expression Sequencing or SCOPESeq.

In the automated cell imaging and RNA library sample preparation system of the present disclosure, single cells are isolated into individual reaction chambers of a microwell array along with a microbead having a plurality of oligonucleotides conjugated on its surface. Each oligonucleotide includes a cell identifying optical barcode sequence that is unique to that bead as well as an RNA binding sequence for RNA capture after cell lysis. The ‘cell identifying optical barcode sequence’ is also referred to herein interchangeably as a ‘cell identifying optical barcode’. The microbeads having the cell identifying optical barcode and RNA binding sequence are also referred to herein interchangeably as ‘mRNA capture beads’ or ‘RNA capture beads’ or ‘microbeads’ or in some instances ‘beads’. The oligonucleotides on the microbeads can include an adapter sequence for sequencing (e.g., for sequencing on Illumina platforms) (otherwise referred to as ‘PCR handle’). The microbeads having the cell identifying optical barcode and the complementary optical hybridization probes of the present disclosure are described in U.S. Pat. Application PCT/US2016/034270, filed on May 26, 2016, and published as WO 2016/191533 and U.S. Pat. Application PCT/US2018/62650, filed on Nov. 27, 2018, and published as WO 2019/104337, which are hereby incorporated by reference in their entireties. The system is configured for flowing optical hybridization probes that are complementary to the cell identifying optical barcodes and labeled with an optical label, such as a fluorophore, onto the microwell array and for obtaining images of the microwells in response to the probes. The system and unique cell identifying optical barcodes and complementary optical hybridization probes facilitate a link between phenotypic imaging of cells resident on the microwell array with single cell whole transcriptome sequencing.

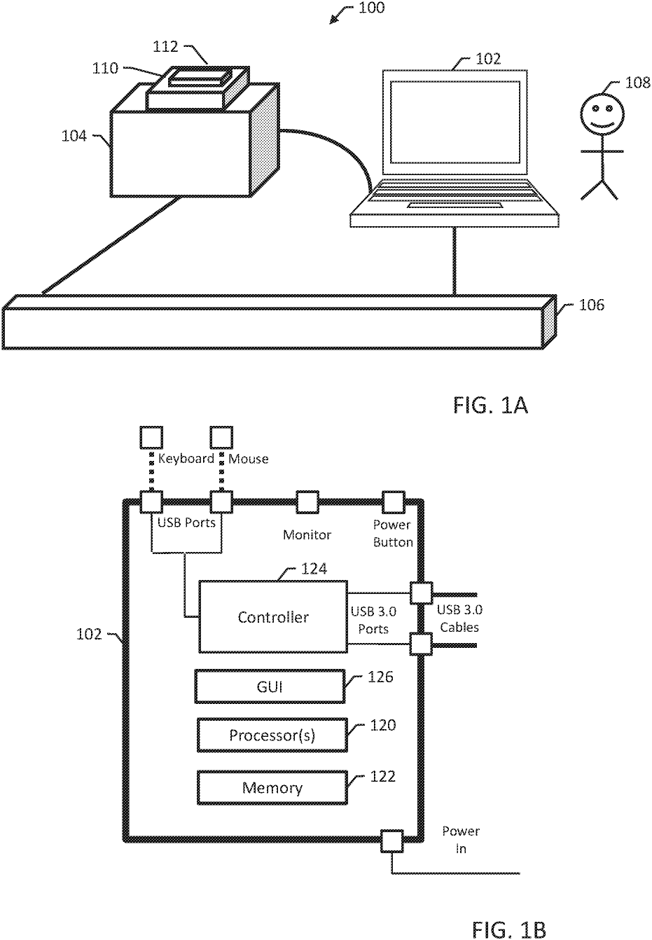

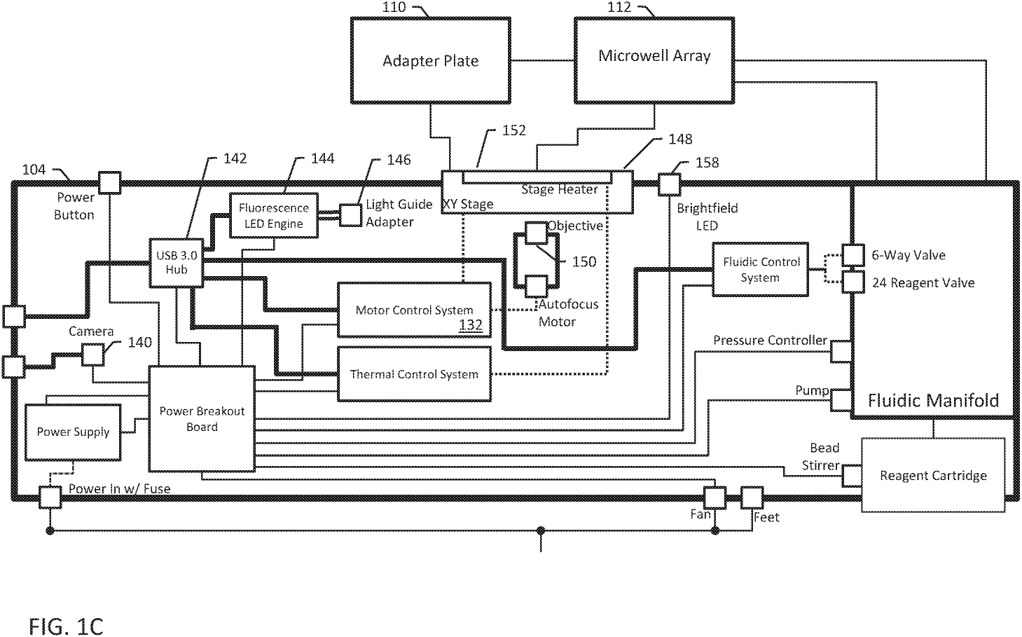

FIGS. 1A-1C are diagrams of an example system 100 for single cell isolation and sample preparation. The system 100 can be used to phenotypically characterize multiple single cells as well as capture and prepare the nucleic acid content for sequencing. Through the use of the RNA capture beads and the unique optical barcode readout from the optical hybridization probes, the system 100 can provide a direct link between live cell images and the sequence of the RNA expressed by the single cell.

FIG. 1A is an overview diagram of the system 100. The system 100 includes a computer subsystem 102, an instrument assembly 104, an experimental environment 106 (e.g., one or more pieces of laboratory equipment such as power supplies and environmental control subsystems), and a user 108. The instrument assembly 104 includes an optional adapter plate for receiving a microwell array 112.

Typically, the user 108 would load the microwell array 112 into the optional adapter plate and place it into the system 100. The system 100 would flow cells from an input reservoir into the microwell array 112 and allow the cells to settle into individual microwells. The system 100 provides scanning, image analysis, and an RNA library sample preparation protocol. Sample preparation can include controlling fluidics and thermal subsystems.

FIG. 1B is a block diagram of the computer subsystem 102. The computer subsystem 102 includes at least one processor 120, memory 122, a controller 124 implemented as a computer program using the processor 120 and memory 122, and a graphical user interface (GUI) 126. For example, the computer subsystem 102 can be a desktop computer with a monitor and keyboard and mouse, or the computer subsystem 102 can be a laptop or tablet computer or any other appropriate device. The computer subsystem 102 is operatively coupled to the instrument assembly 104, e.g., by universal serial bus (USB) cables. In some examples, the computer subsystem 102 is integrated into the instrument.

The controller 124 is programmed for identifying microwells that each contain a single cell. The controller 124 can be programmed for identifying other relevant features in images of the cells within the microwells.

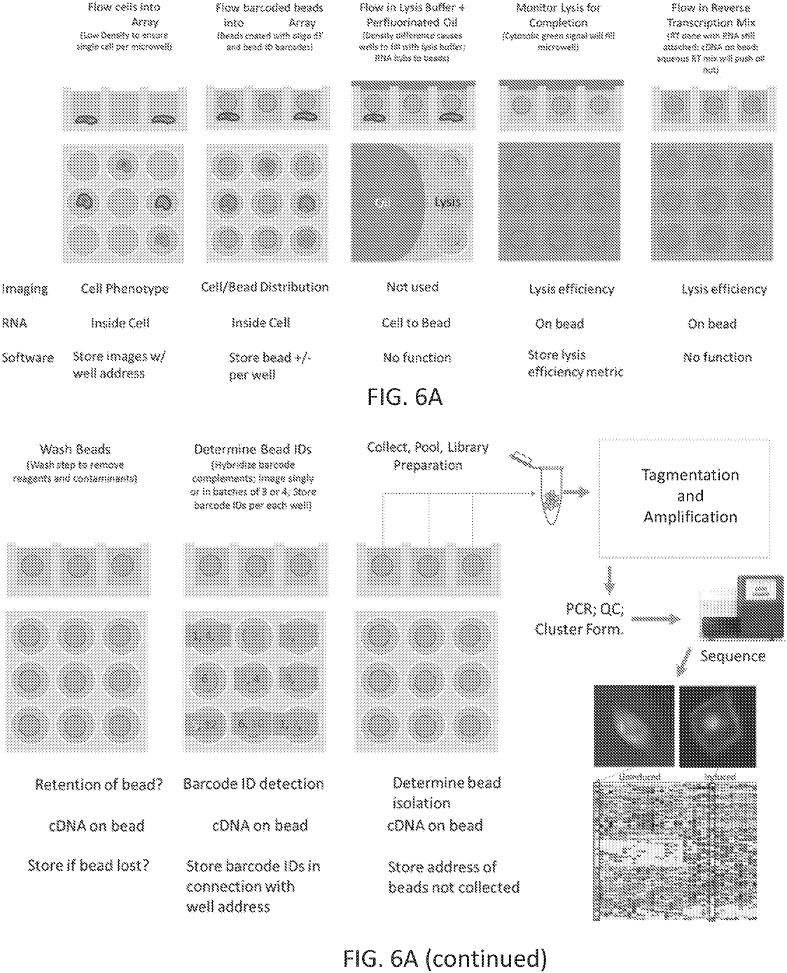

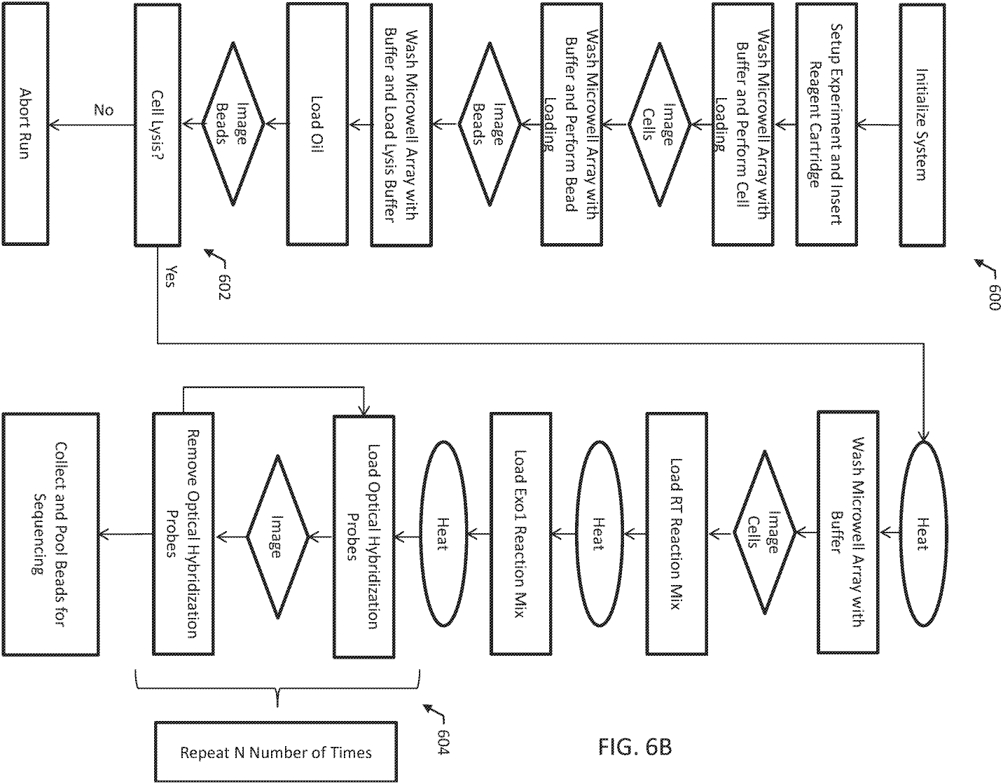

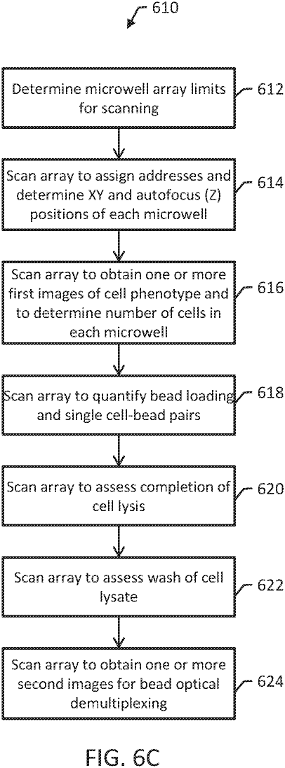

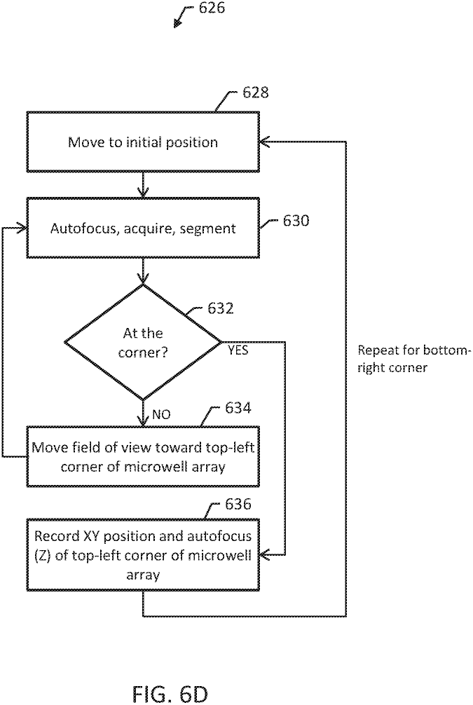

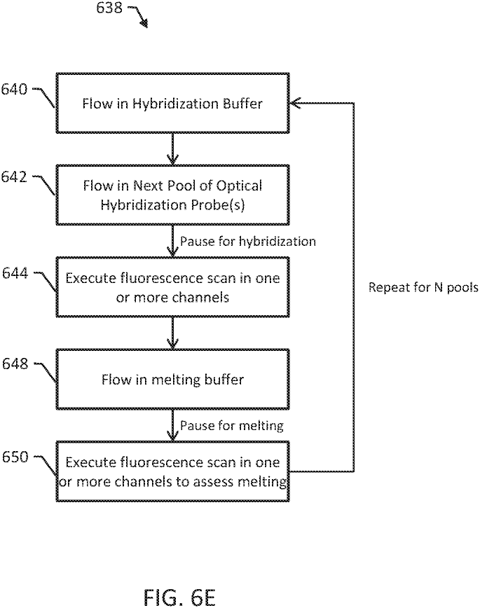

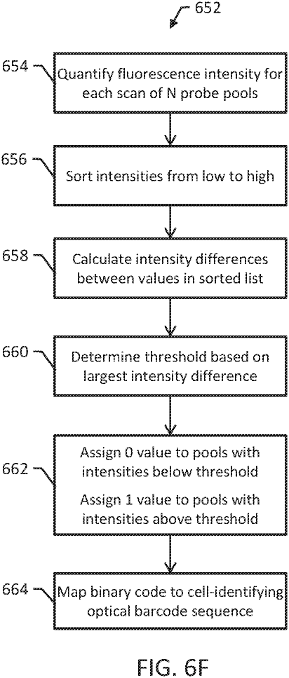

The controller 124 is programmed for causing the system 100 to automate the SCOPESeq process as described below with reference to FIGS. 6A-6B. For example, the controller 124 can be programmed to store a record for each microwell in the array 130 and to associate, with each microwell record, one or more images of the microwell and identifying features of the microwell contents such as a phenotypic information of the cell and optical barcode readout (e.g., a fluorescent signal) associated with a microbead residing in the microwell in the presence of the complementary optical hybridization probe.

FIG. 1C is a block diagram of the instrument assembly 104. The instrument assembly 104 can include various components for imaging individual microwells 130 on the microwell array 130. For example, the instrument assembly 104 can include a power breakout board 138 and a motor control system 132 for controlling various motors. The motor control system 132 can contain, e.g., TTL and shutter functions that allow the controller 124 to control or address various components of the instrument assembly 104.

The instrument assembly 104 can include a digital camera 140 or other appropriate imaging device, a communications hub (e.g., USB Hub 142), a fluorescence light emitting diode (LED) engine 144, and a light guide 146. The light guide 146 delivers the fluorescence excitation light from the LED engine to the microscope. Alternate configurations include a fiber optic bundle or even direct coupling of the LED engine to the microscope optical train.

The fluorescence LED engine 144 can include multiple narrow-band LEDs configured to illuminate the microwell array 112 by way of the light guide adapter 146.

The instrument assembly 104 includes a microscope subsystem (e.g., an internal inverted microscope) including a motorized XY stage 148 and an autofocus motor 150 configured for translating a microscope objective 152. Typically, the camera 140 and the fluorescence LED engine 144 and microscope subsystem are arranged in an epi-fluorescence configuration. The instrument assembly 104 includes a bright-field LED 158 for illuminating the microwell array 112 during imaging.

The instrument assembly 104 includes a microfluidic subsystem and a thermal subsystem 152. The thermal subsystem 152 can include, for example, a stage heater on the XY stage 148 and a thermal control system for controlling the stage heater. The microfluidic subsystem includes a pump, a pressure controller, and a fluidic manifold. The microfluidic subsystem includes various appropriate valves, for example, a 6-way valve and a 24-reagent valve for application of reagents from a reagent cartridge. The controller 124 is programmed to control the microfluidic subsystem and the thermal subsystem to automate the SCOPE-seq process as described further below with reference to FIGS. 6A-6B.

In some examples, the microfluidic subsystem is configured for microfluidic flow control of, e.g., eighteen different reagents to fulfill the biochemical reactions of the SCOPEseq process. In addition, various flow rates can be used from, e.g., 10 µL/min to 200 µL/min that are controlled within 5 µL/min of the set point.

The microfluidic subsystem can include a flow rate unit configured for accurate and simple flow rate measurement capability that is compatible with a variety of reagents that range from organic to aqueous to fluorinated oil. The unit can have measurement feedback capabilities to the flow rate controller that will provide accurate flow rate control throughout the microfluidic subsystem.

The microfluidic subsystem can include a flow control unit configured for pulse-free flow to facilitate fluidic movement without cell shear stress. This unit can have a millisecond response time between reagent switching and bubble-free fluidic flow.

The microfluidic subsystem can include valving units, e.g., two sets of unique valving units. First, a multi-way bidirectional valve that can multiplex with a second multi-way valve can be used to switch between different reagents to flow into the microchip. These switch units have millisecond response time to rapidly adjust to new reagent flow. This will provide appropriate flow responses for microwell sealing with fluorinated oil. Second, multi-way valves may be used to direct reagents from the output port of the microchip to sample collection or waste reservoirs. The multi-way valving units will also eliminate any hydrostatic flow, providing a pressurized flow cell which will be necessary for imaging and heating.

The microfluidic subsystem can include pressurized reagent reservoirs. For instance, reagent cartridges can be used that ensure appropriate sealing of the reagents, as well as maintaining sufficient pressurized environments for fluid flow into the microfluidic subsystem.

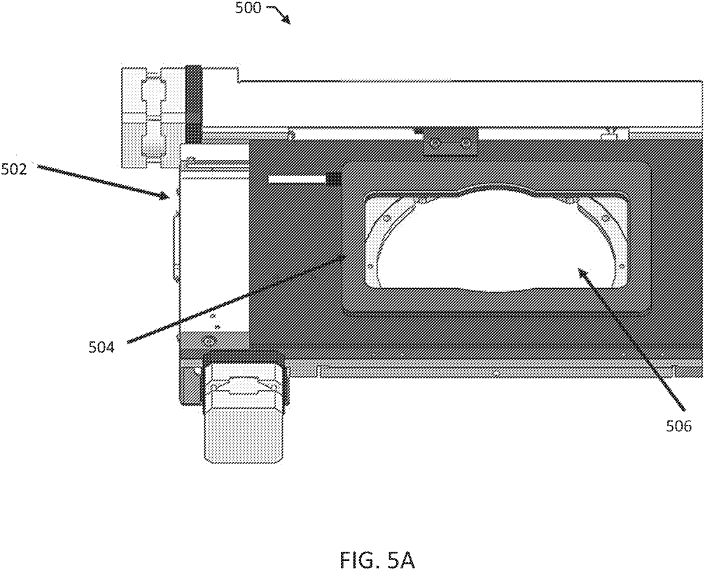

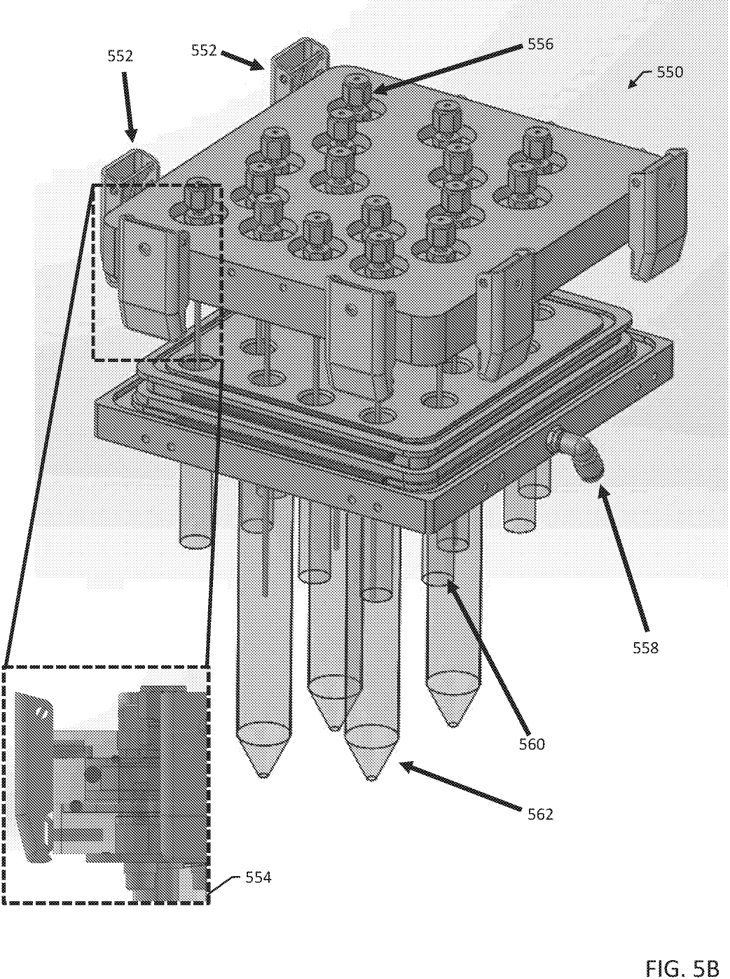

The thermal subsystem can include one or more Peltier units that can heat and cool throughout a workflow to provide constant temperature control when necessary to facilitate appropriate conditions for various biochemical assays. In some examples, the thermal subsystem includes a proportional, integral, derivative (PID) thermal control unit, e.g., with accuracy with 1° C., to facilitate proper PID feedback to the Peltier units to set and control appropriate assay temperatures. In some examples, the thermal subsystem includes a stage heater integrated with the XY stage, e.g., as shown in FIG. 5A. The thermal subsystem can be used, e.g., to accelerate lysis, facilitate the RT and EXO1 processes, and in some cases to promote melting of the optical probe hybridization.

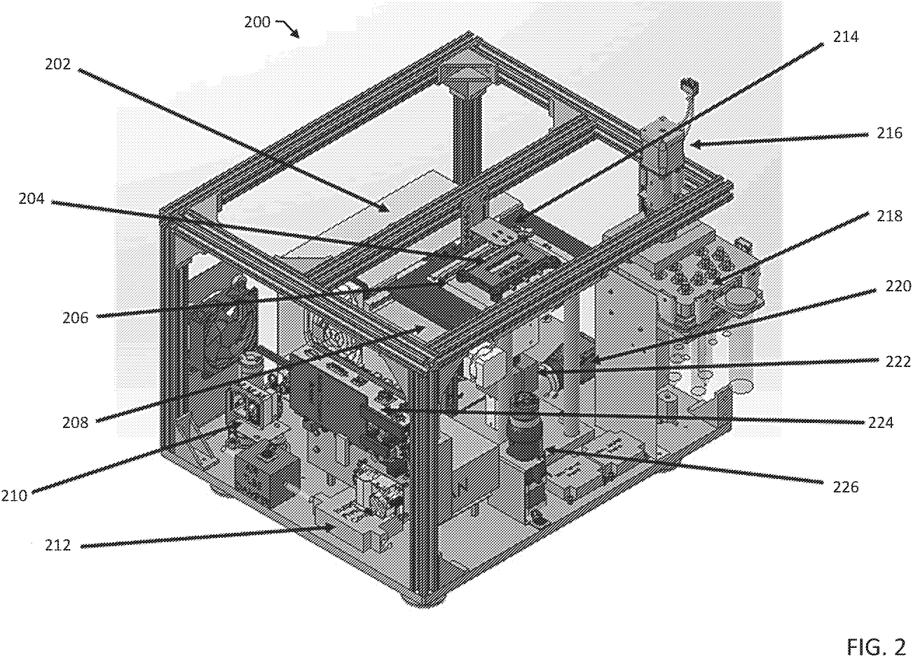

FIG. 2 illustrates an example mechanical device 200 for the system 100. The device 200 includes a fluorescence engine 202, an adapter plate 204, an array stage 208, a stage heater 206, and an XYZ stage control system 224. The device 200 includes a pump 210 and a pressure controller 212. The device 200 includes a bright-field module 214, a fluidic control device 216, and reagent cartridge 218. The device 200 includes a camera 220 and an optical stack 222. The device 200 includes electronics (e.g., a power supply) and a fluidic control device 226.

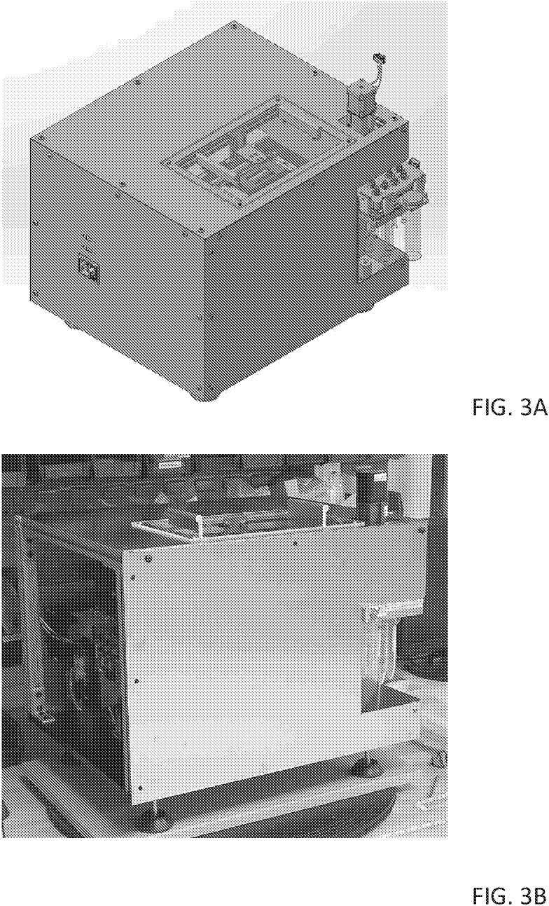

FIG. 3A shows a 3D model of the device 200 with enclosure. FIG. 3B shows an example implementation of the device 200 with a side cover removed to illustrate internal components.

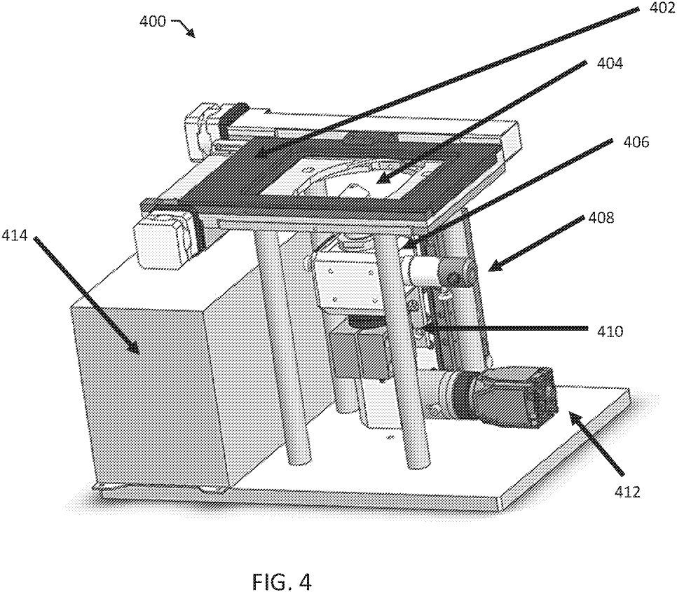

FIG. 4 shows a 3D model of an example imaging subsystem 400. The imaging subsystem 400 includes an XY stage 402, an objective lens 404, and a filter set 406. The imaging subsystem 400 includes a liquid light guide entrance 408, a focus drive 410, and a camera 412. The imaging subsystem 400 includes an LED engine 414, which can include, e.g., an LED controller, LEDs, combining optics, and a light guide exit port.

FIG. 5A is a top-down view of an example thermal subsystem 500. The subsystem 500 includes an XY stage 502 and a stage heater 504 for heating the microwell array 502. The subsystem 500 can include a glass component 506 to allow imaging of a sample while applying heat to the sample. In some examples, a computer control subsystem is configured to automate control of the subsystem 500.

FIG. 5B is block diagram of an example subsystem 550 including an interface between the reagent cartridge and the fluidic manifold. The subsystem 550 includes pressure clasps 552 for securing the subsystem 550; an example pressure clasp is illustrated in a detail view 554. The subsystem 550 includes multiple fluidic lines 556, a single pressure input 558, and different sized reservoirs 560 and 562.

FIGS. 6A-6B are flow diagrams of an example method for preparing an RNA library from a single cell for sequencing using the automated system, as well as capture of unique optical barcode readout for use in the association of single cell phenotypic and gene expression sequence data.

Cells are first flowed onto the microwell array to provide a random distribution with a relatively large fraction of cells residing singly in a given microwell. Cells can be imaged on the microwell array at this time to collect phenotypic data as well as to determine those microwells containing a single cell. Cells can be stained in any manner as would be understood by those of ordinary skill in the art to facilitate collection of phenotypic information. Microbeads are then flowed into the chamber. The size of the wells and size of the beads are harmonized to ensure only one bead can reside in a given microwell, and a concentration of beads is used such that greater than, e.g., 75%, 80%, 85%, or 95% of wells contain a single bead.

Lysis buffer can then be flowed onto the microwell array, immediately followed by perfluorinated oil. The oil effectively “seals” each microwell from aqueous cross-contamination. RNA is then captured by the beads after lysis and reverse transcriptase mix can then be flowed onto the microwell array. At this point, the RNA captured on the beads has been reverse transcribed to cDNA and the complementary optical hybridization probes can be flowed in and imaged to determine bead-cell linkage. The data association between the cell identifying optical barcode for the microwell position and the first image at the position is stored by the system and used to link the cell images taken prior to library preparation to the genomic (or transcriptomic) data generated during sequencing.

FIG. 6B is a flow diagram of the process 600 carried out by the system. FIG. 6B illustrates the automated verification 602 of cell lysis by image analysis of images of the microwells. FIG. 6B also illustrates the method for associating single cell images with unique optical barcode readout 604 by loading a plurality of the optical hybridization probes, imaging the microwell array N number of times, and performing image analysis to determine matches between the optical hybridization probes and the cell identifying optical barcodes for each microwell position. The method includes storing the data association between the cell identifying optical barcode for the position and the first image of the microwell contents that position captured prior to loading of the beads.

FIGS. 6C-6F illustrate processes that may be carried out by the system in performing the process 600 shown in FIG. 6B.

FIG. 6C is a flow diagram of a process 610 for the imaging performed by the system. The process 610 includes determining microwell array limits for scanning (612). The process 610 includes scanning the array to assign addresses to array positions and to determine XY and autofocus (Z) positions of each microwell (614). The process 610 includes scanning the array to obtain one or more first images of cell phenotype and to determine a number of cells in each microwell (616). The process 610 includes scanning the array to quantify bead loading and single cell-bead pairs (618). The process 610 includes scanning the array to assess completion of cell lysis (620). The process 610 includes scanning the array to assess the wash of cell lysate (622). The process 610 includes scanning the array to obtain one or more second images for bead optical demultiplexing (624).

FIG. 6D is a flow diagram of a process 626 for the determination of chip scan limits. The process 626 includes moving a current position of the field of view to an initial position (628) and autofocusing, acquiring an image, and segmenting the image (630). The process 626 includes determining if the current position is at a corner (632). If the current position is not at a corner, the process 626 includes moving the current position towards a top-left corner of the microwell array (634) and repeating until the corner is found. When the corner is found, the process 626 includes recording the XY position of the corner and autofocusing (Z) the top-left corner of the array. The process 626 includes repeating for the bottom-right corner.

FIG. 6E is a flow diagram of a process 638 for system control of probe hybridization and melting. The process 638 includes flowing in a hybridization buffer 540. The process 638 includes flowing in a next pool of optical hybridization probe(s) and then pausing for a programmed length of time to allow for hybridization (642). The process 638 includes executing a fluorescence scan in one or more channels (644). The process 638 includes flowing in a melting buffer and pausing for a programmed length of time to allow for melting (648). The process 638 includes executing a fluorescence scan in one or more channels to assess melting (650). The process 638 includes repeating steps 640, 642, 644, 648, and 650 for each of N pools of optical hybridization probes, until the unique cell identifying optical barcode sequence attached to each bead can be decoded.

FIG. 6F is a flow diagram of a process 652 for optical demultiplexing. The process 652 is performed for each bead-containing microwell and fluorescence channel. The process 652 includes quantifying fluorescence intensity for each scan of N probe pools (654). The process 652 includes sorting intensities from low to high (656). The process 652 includes calculating intensity differences between values in the sorted list (658). The process 652 includes determining an intensity threshold based on the largest intensity difference (660), e.g., by selecting a threshold between the two intensities bounding the largest intensity difference. The process 652 includes assigning a 0 value to pools with intensities below the threshold intensity and assigning a 1 value to pools with intensities above the threshold intensity (662). The process includes mapping the binary code yielded by the 0 and 1 values to a cell-identifying optical barcode sequence (664).

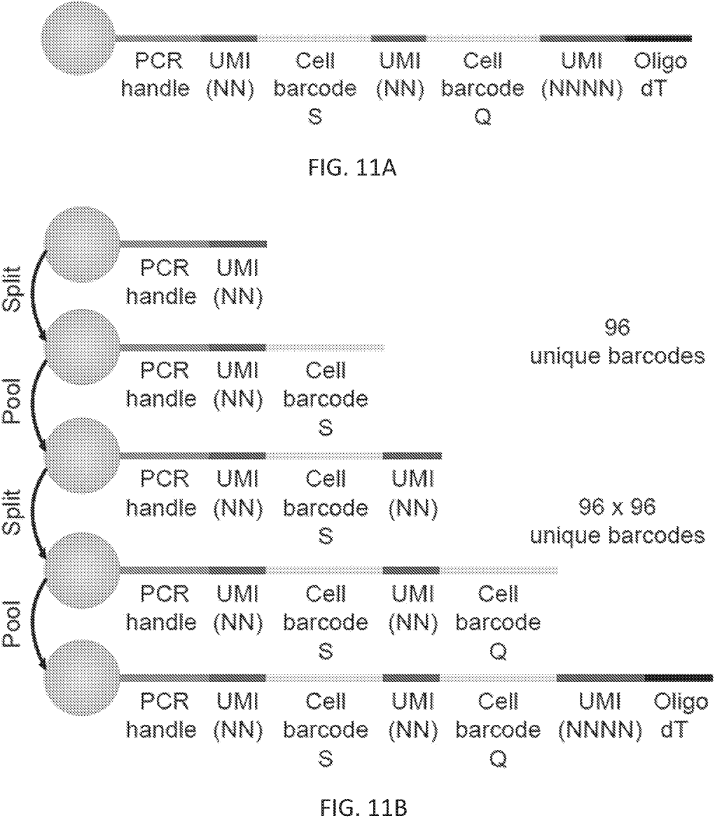

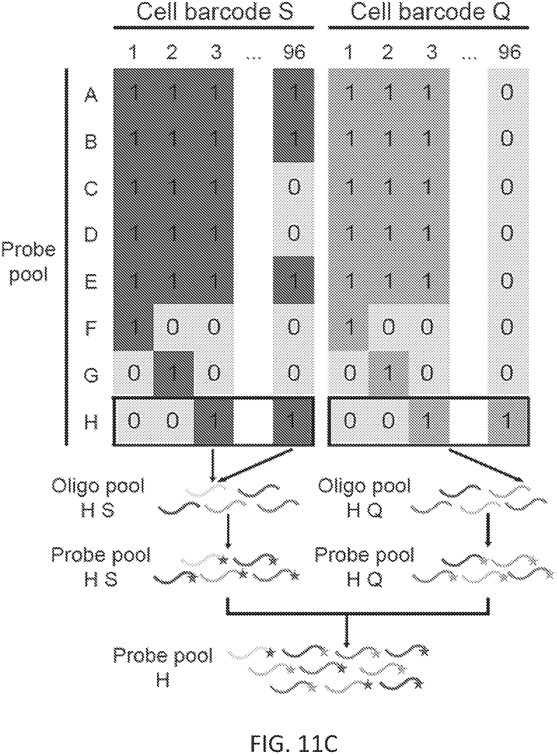

For example, consider the following discussion of an example method for optical demultiplexing described in Example 5. In this example method, 96 out of 256 possible binary codes are used (see FIGS. 11A-11C and Examples 2 and 3 for the design and synthesis of the beads and optical hybridization probes). In this embodiment, the number of sequenced cell identifying optical barcodes (~1,000 cells per experiment on the microwell array of the automated system) are much fewer than the total 9,216 possible barcodes (i.e. 96 X 96 = 9,216 unique barcodes). Therefore, an error in optical decoding would mainly result in assigning the bead an unmappable binary code, or a cell identifying optical barcode that does not appear in the sequencing data. Both kinds of misassignments further lead to the failure of linking imaging and sequencing data sets rather than incorrect linking. Thus, a more accurate optical decoding method would give a higher fraction of linked imaging and sequencing data.

To decode the cell barcode sequences from imaging, a ‘cycle-by-cycle’ method can be used, which calls the binary code for each bead based on the bimodal distribution of intensity values across all beads in each hybridization cycle. This method works well when the bead fluorescence intensity values of the ‘one’ state population are well separated from that of the ‘zero’ state population. However, because the beads exhibit auto-fluorescence at shorter wavelengths, the two populations are not clearly separated in the Cy3 emission channel.

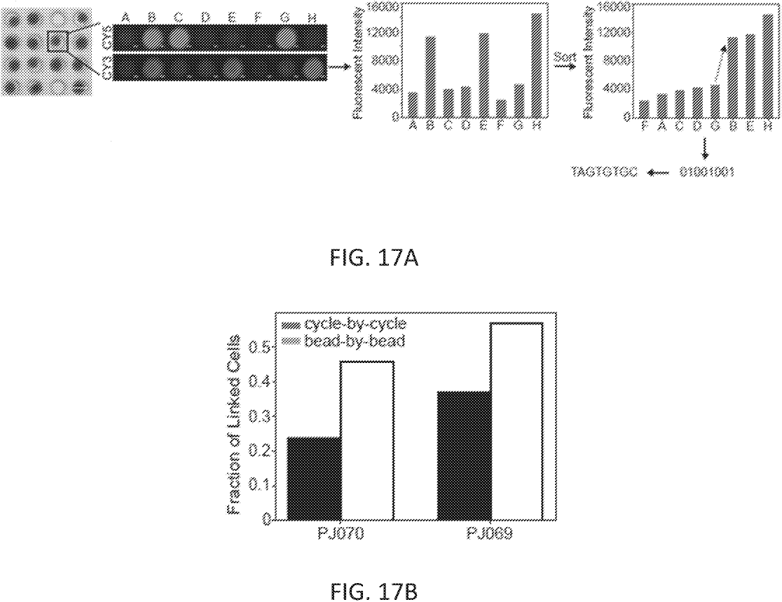

To accurately decode the cell barcode sequences from imaging, the system can utilize a modified ‘bead-by-bead’ fluorescence intensity analysis strategy. The cell barcode sequences of each bead are determined by sorting the eight intensity values in ascending order, calculating the relative intensity change between each pair of adjacent values, establishing a threshold based on the largest relative intensity change to assign a binary code, and mapping the binary code to the actual cell barcode sequence (see FIG. 17A). For those unmappable binary codes, the binary code is repeatedly re-assigned based on the next largest relative intensity change until the code can be successfully mapped to a cell barcode sequence. Since this method decodes each bead independently, it can give better results when the ‘one’ and ‘zero’ intensity states are poorly separated.

Example 5 describes a comparison of the cycle-by-cycle and bead-by-bead methods. In dataset PJ070 and PJ069, 46% and 57% scRNA-seq profiles are linked with cell images using the ‘bead-by-bead’ method in comparison to only 24% and 37% using the ‘cycle-by-cycle’ method. In both datasets, at least a 20% increase is observed in the fraction of linked cells with the ‘bead-by-bead’ method (FIG. 17B), which suggests that the ‘bead-by-bead’ method is more suitable for cell identifying optical barcode sequence decoding by image analysis.
Cycle-by-cycle
   The cycle-by-cycle method was modified from the stage-by-stage decoding method
      For each cycle and each fluorescent channel;
      Get N log transformed average intensity values;
      Compute an intensity histogram using 50 bins;
      Determine the median intensity value M, and identify the highest bin with intensity values smaller than M as B₁ and the highest bin with intensity values greater than M as B₂;
      Identify the lowest bin B₃ with intensity values between B₁ and B₂;
      Get the medium intensity value I of bin B₃, then assign 0 to intensity values smaller than I and assign 1 to intensity values greater than I.
      Referto the binary code table. If the code assigned is in the table, then return the corresponding cell barcode sequence.
Bead-by-bead
   The bead-by-bead method was modified from the core-by-core decoding method
      For each bead and each fluorescence channel;
      Get eight average fluorescence intensity values x₁,x₂,...,x₈;
      Let y₁,y₂,...,y₈ be the sorted values;
      Let fₙ = (yₙ₊₁ - yₙ)/yₙ, n = 1,2,...,7 be the relative intensity fold change between neighbor sorted values;
      Determine the largest fold change N = argmax(fₙ), then assign 0 to values n to y₁,y₂,...,y_{N} and assign 1 to values y_{N+1},y_{N+2},...,y₈;
      Refer to the binary code table. If the code assigned in step 4 is in the table, then return the corresponding cell barcode sequence;
      Otherwise, remove f_{N} from list { fₙ } and repeat step 4, 5, until a corresponding cell barcode sequence is returned or the list {fₙ} is empty.

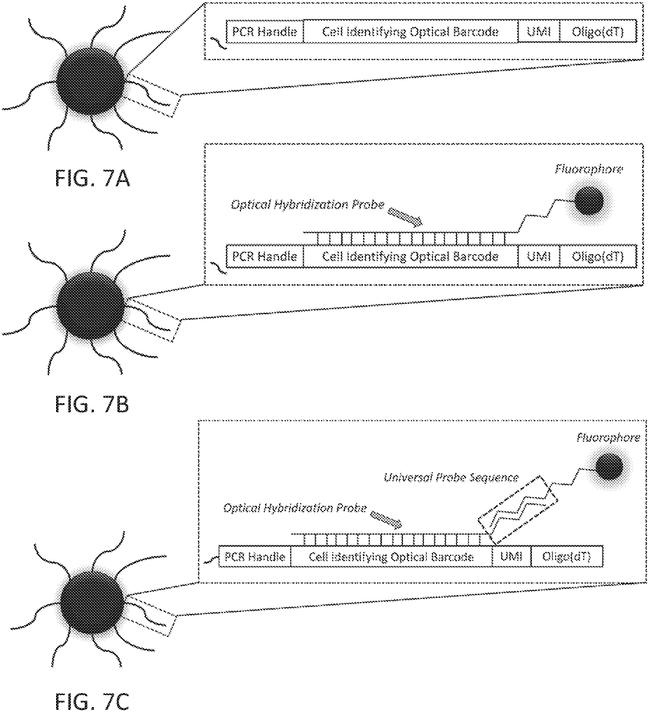

FIGS. 7A-7C display the binding of the optical hybridization probes to the complementary cell identifying optical barcode sequences on the microbeads. FIG. 7A depicts an example of the microbeads with attached oligonucleotides including an adapter sequence, a cell identifying optical barcode sequence that is unique for the bead, a unique molecular identifier (UMI) sequence, and oligo-dT for RNA capture. FIG. 7B depicts binding through hybridization of an optical hybridization probe to its complementary cell identifying optical barcode sequence of the microbead, with a fluorophore directly attached to the probe for identification during imaging. FIG. 7C depicts an alternate embodiment in which the optical hybridization probe is made up of two separate molecules in which the first contains a sequence complementary to a cell identifying optical barcode and a universal binding sequence, and the second contains a sequence that is complementary to the universal binding sequence and also contains an optical label, such as a fluorescent label, to facilitate simple and cost-effective synthesis of the fluorescent probes. In this case, the first molecules of the optical hybridization probes are flowed onto the microwell array, followed by the second molecules, followed by imaging, and removal of both probes. A plurality of hybridization probes can be flowed onto the microwell array of the system at a time in order to minimize the number of N repeats as depicted in 604 of FIG. 6B.

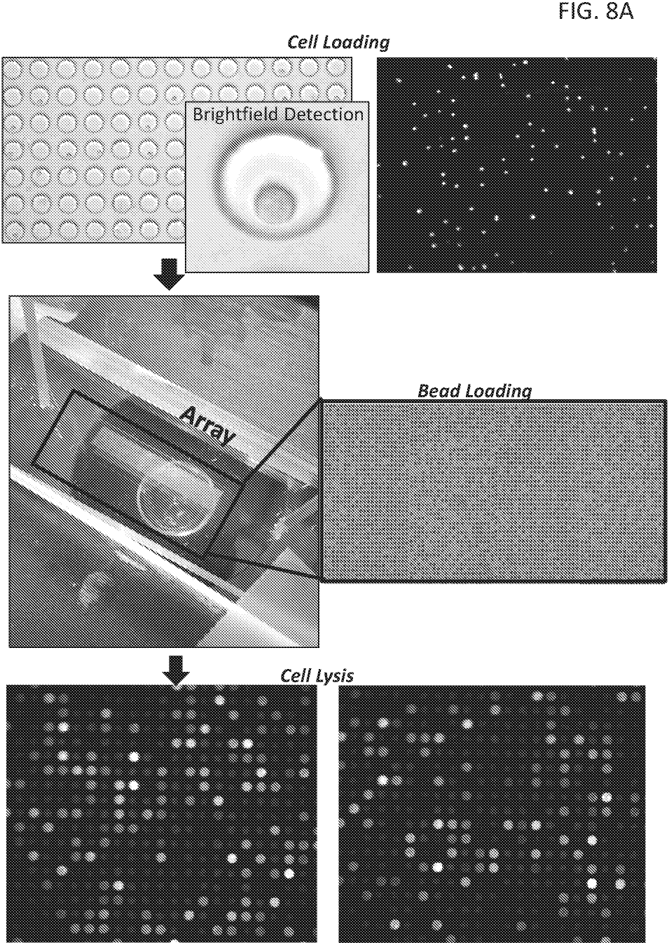

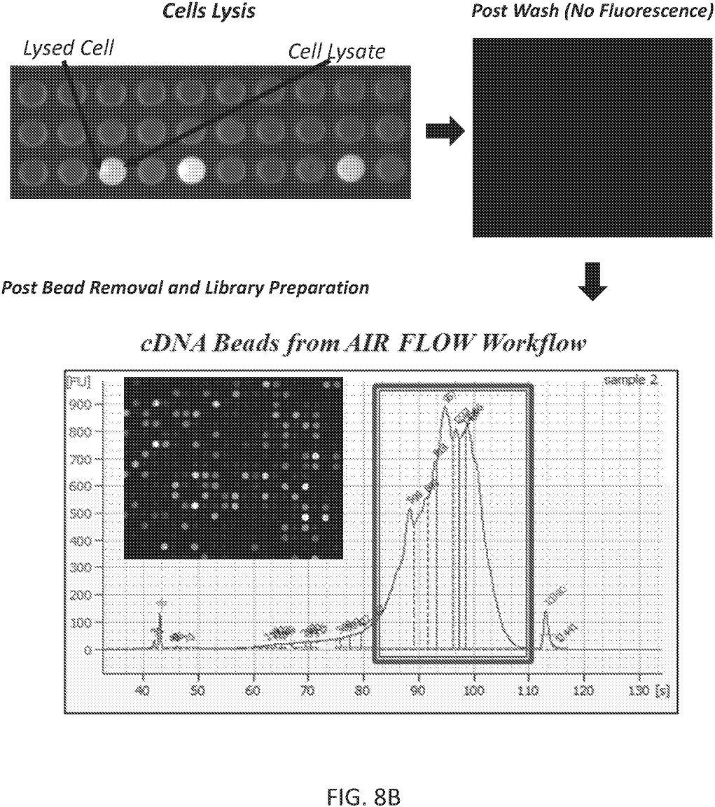

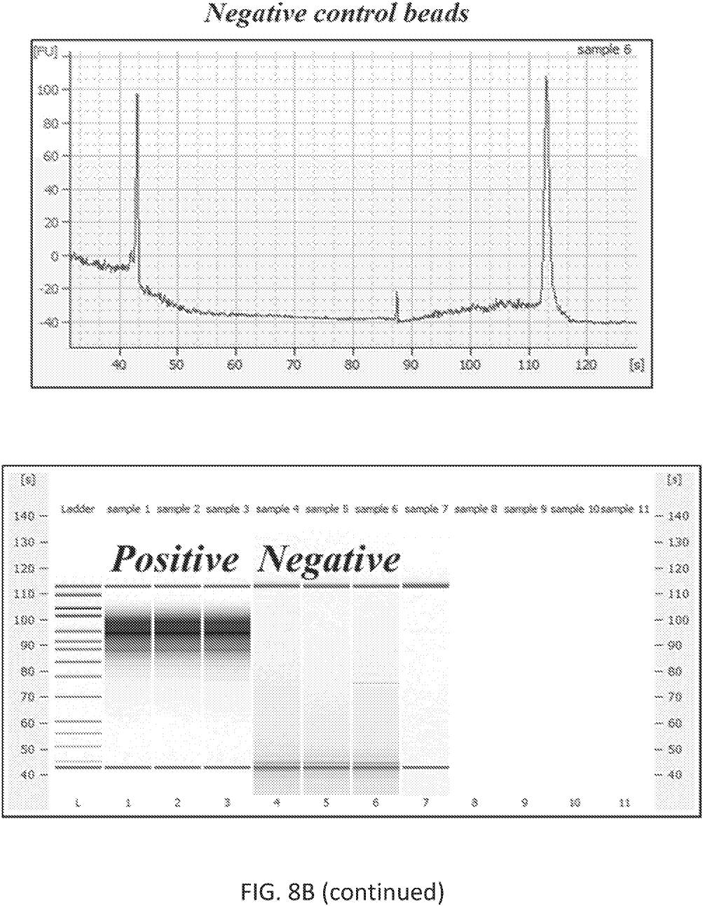

FIGS. 8A-8B display data and images for cell and bead loading of the system 100 and method of the present disclosure followed by cell lysis in the individual wells. 10% cell loading (~1000 cells in a 10,000 microwell array) using the fluidics subsystem is depicted followed by fluorescence imaging of the fluorescently labeled cells in which the image reveals the microwells containing a single cell. The cells can be loaded to obtain a large majority of single cells per microwell. The beads are loaded using the fluidics subsystem at a higher density than the cells and can be loaded to maximize the number of single cell-single bead pairs. A microwell array of the system is shown in FIG. 8A with 85% bead loading (~8,500 beads in 10,000 microwell array). Cell lysis can be performed after bead loading where a lysis buffer is flowed onto the microwell array using the fluidics subsystem which is rapidly followed by flowing in an oil to seal the microwells. As depicted in FIG. 8A, the cells begin as little dots under the fluorescent detection, but as the cell is lysed the dye diffuses throughout the microwell indicating that lysis was done successfully. In addition, the fluorescent signal remains within the wells indicating that no cross contamination between the microwells is occurring (i.e. the oil covered the wells properly).

FIG. 8B displays the lysis in greater detail, where the remnants of the cell can be seen, with the lysate filling the microwell. Image processing by the system 100 can automatically detect successful completion of lysis by analyzing the dye diffusion.

When the oil is washed out after lysis, the lysate is completely removed from the microwells, showing a dark response while imaging. This QC step confirms that the microwell array has been washed successfully and that the RT mix has the ability to be in contact with every bead (the RNA is attached to the beads at this point and therefore cannot be washed out or result in cross contamination). After completion of the system 100 operations, the beads are removed and can be pooled for further cDNA library preparation including DNA amplification followed by nucleic acid sequencing. An electropherogram in FIG. 8B displays cDNA prepared with the system 100 and method of the present disclosure having the correct length and concentration of cDNA required for sequencing.

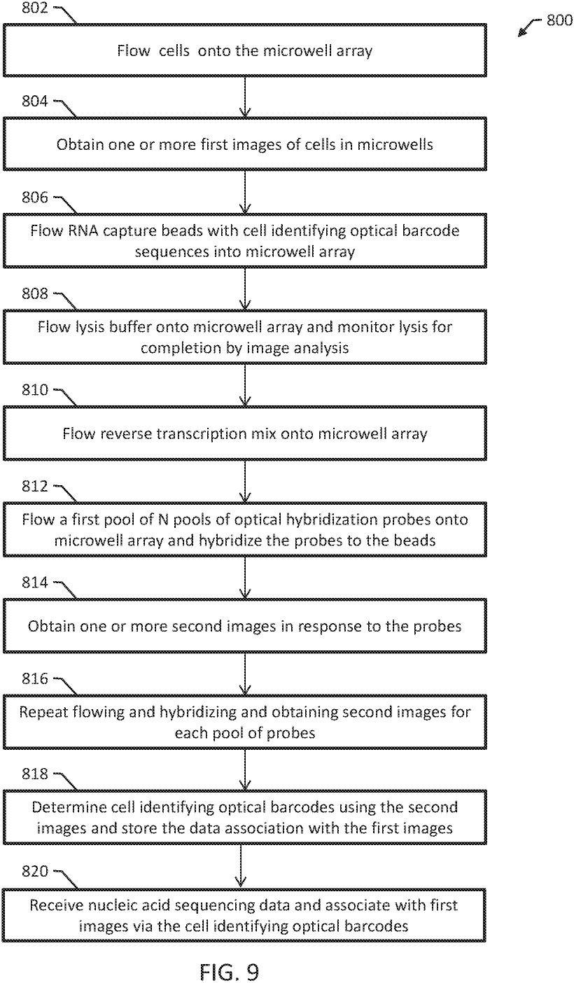

FIG. 9 is a flow diagram of an example automated method 800 for associating single cell imaging data with RNA transcriptomics. The method 800 can be performed by a control subsystem, e.g., the controller 124 of FIG. 1.

The method 800 includes flowing cells onto the microwell array of the system 100 (802) and obtaining, for each position in the microwell array, one or more first images at the position using an imaging subsystem (804). The first images can depict, e.g., cells loaded into the microwells of the array and information about the phenotype of the cells. Each image is associated with a corresponding position of the microwell in the array. The position can be specified, e.g., as an X-Y coordinate on the microwell array. In some examples, the method 800 includes determining, for each position, a number of cells depicted in a microwell corresponding to the position using the first image of the position. This allows for downstream elimination of data for microwells containing more than one cell.

The method 800 includes flowing, using a fluidics subsystem, RNA capture beads having attached cell identifying optical barcode sequences onto the microwell array (806). The method 800 includes flowing, using the fluidics subsystem, a lysis buffer onto the microwell array and imaging, using the imaging subsystem, the microwell array and performing image analysis to monitor lysis for completion within the microwells (808). The method 800 includes flowing, using the fluidics subsystem, reverse transcription mix onto the microwell array after determining completion of lysis based on performing image analysis (810).

The method 800 includes flowing, using the fluidics subsystem, a first of N pools of optical hybridization probes onto the microwell array and hybridizing the probes to the beads located therein having a complementary nucleotide sequence in the cell identifying optical barcode sequence attached thereto (812). The method 800 includes obtaining, for each position of the plurality of positions, one or more second images to quantify a fluorescent intensity at the position using the imaging subsystem, each of the one or more second images used to create a binary code depicting a match or a lack of a match between at least one of the optical hybridization probes and the cell identifying optical barcodes (814). A match can be identified where a sufficient intensity of light is identified in an image of a microwell containing a microbead after flowing the optical hybridization probe.

The method 800 includes repeating the flowing and hybridizing step and obtaining the one or more second images step for each of the N pools of probes (816).

The method 800 includes determining, by mapping the binary code for each of the N pools of probes to the cell identifying barcode sequence, for each position of the plurality of positions, the cell identifying optical barcode for the position and storing a data association between the cell identifying optical barcode for the position and the first image at the position (818). For example, determining the cell identifying optical barcode can comprise a digital value formatted such that each bit position in the value corresponds to a match or a lack of a match between an optical hybridization probe or a pool of optical hybridization probes and a cell identifying optical barcode.

In the method 800, microbeads are removed from the microwell array for sequencing. The method 800 includes storing, for each position of the plurality of positions, after receiving nucleic acid sequencing data for each cell identifying optical barcode, a data association between the nucleic acid sequencing data, the cell identifying optical barcode, and the first image associated with the cell identifying optical barcode (820).

The method 800 can include displaying a graphical user interface (GUI) for controlling various aspects of the process. For example, the GUI can provide controls for starting and stopping a run. The GUI can provide images of specified cells at various stages of a run. The GUI can present status reports during a run.

In some examples, the method 800 includes recovering the microbeads. For example, recovering the microbeads can include inverting the chip to allow the beads to settle by gravity into the flow channel. Recovering the microbeads can include flowing in a high-density fluid that will “float” the beads up into the flow channel. Recovering the microbeads can include pulsing the flow to agitate the beads out of their wells into the flow channel. Recovering the microbeads can include sonicating the beads to agitate the beads out of their wells into the flow channel. Recovering the microbeads can include chemically or optically cleaving the cDNA from the beads to allow it to be collected while the beads themselves are left behind.

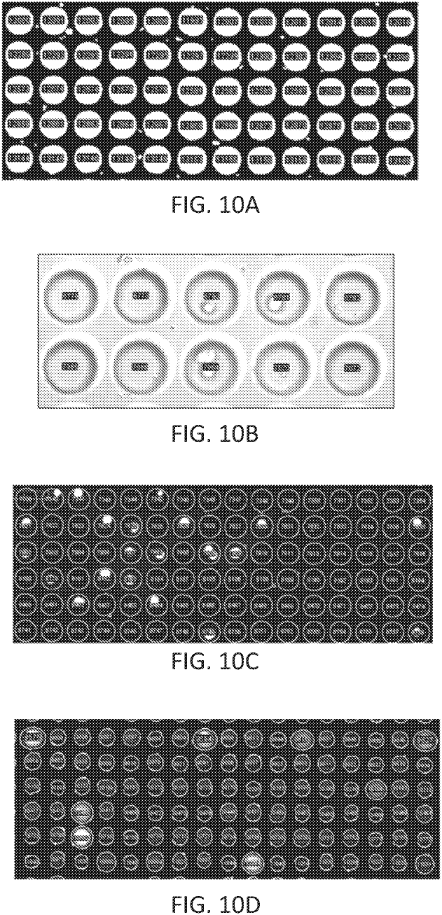

FIGS. 10A-10D illustrate image analysis. FIG. 10A shows a binary image of segmented and labeled microwells. FIG. 10B shows a bright-field image of cells in microwells. FIG. 10C shows a fluorescent image of live-stained cells. FIG. 10D shows a fluorescent image of the microwells in FIG. 10C after cell lysis.

FIG. 11A is a diagram illustrating one embodiment of the cell identifying optical barcode sequences attached to the RNA capture beads that allow for optical decoding to identify an image of a given cell co-encapsulated with the bead in the microwell array. In this example, the cell barcode contains two 8-nucleotide sequences, each of which is a member of a pool of 96 sequences. An 8-nucleotide random sequence is dispersed into three parts and serves as both a unique molecular identifier (UMI) and a spacer between other functional sequences on the bead. The oligonucleotides on all beads share two common sequences - a universal PCR adapter on the 5′-end and oligo(dT) on the 3′-end for RNA capture and cDNA amplification. The oligonucleotides can be synthesized by split-pool, solid-phase synthesis as described in Example 2 and illustrated in FIG. 11B. Beads are pooled together to add common sequences and random UMIs and are split into 96 reactions to add one of the 96 cell barcode sequences. After two rounds of split-pooling, a total of 96² = 9,216 cell barcodes are generated. To generate cDNA from cells in the methods using the automated system, the cells are co-encapsulated with these beads, the cells are lysed after which cell RNAs are captured on the beads by hybridization, and then the RNA is reverse transcribed.

To link cellular imaging with scRNA-seq from the same cell, the cell identifying optical barcode sequence on each bead is identified in the microwell array by sequential fluorescent probe hybridization. Each cell barcode (i.e. “S” and “Q” in FIG. 11A) corresponds to a unique, pre-defined 8-bit binary code in the cell identifying optical barcode sequence. Each bit of the binary code can be read out by one cycle of probe hybridization, where the presence or absence of a hybridized probe indicates one or zero, respectively. The two parts of the cell identifying optical barcode sequence can be decoded simultaneously using two sets of differently colored fluorescent probes. To realize this decoding scheme, a pool of fluorescent probes is generated for each cycle of hybridization (see Example 3). All probes that can be hybridized to the cell barcode sequence marked ‘1’ in the corresponding binary code are pooled and conjugated with fluorophores, such as, for example, Cy5 or Cy3. Distinct fluorophore-conjugated probes against the two 8-nucleotide sequences comprising the cell identifying optical barcode sequence are then pooled together to form the final probe pool (FIG. 11C. Thus, all possible cell barcode sequences can be decoded by eight cycles of two-color probe hybridization. This approach is compatible with higher speed imaging, leading to higher throughput.

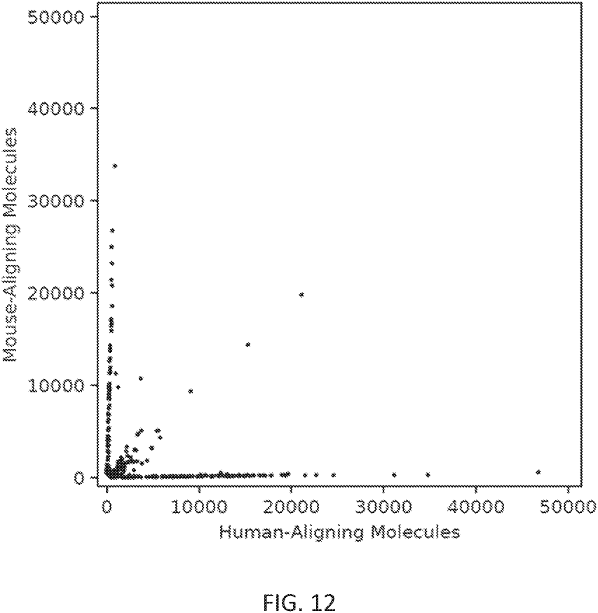

The accuracy of the sequencing data that can be obtained from cDNA library preparation using the automated instrument is illustrated in FIGS. 12-14. In this example method, experiments were performed with mixed human (U87) and mouse (3T3) cells labeled with two differently colored live staining dyes as described in Example 1. The sequencing data resulting from the 5 experiments is shown in Table 1. The data show the automated system can produce high purity cDNA libraries from multiple cell types.

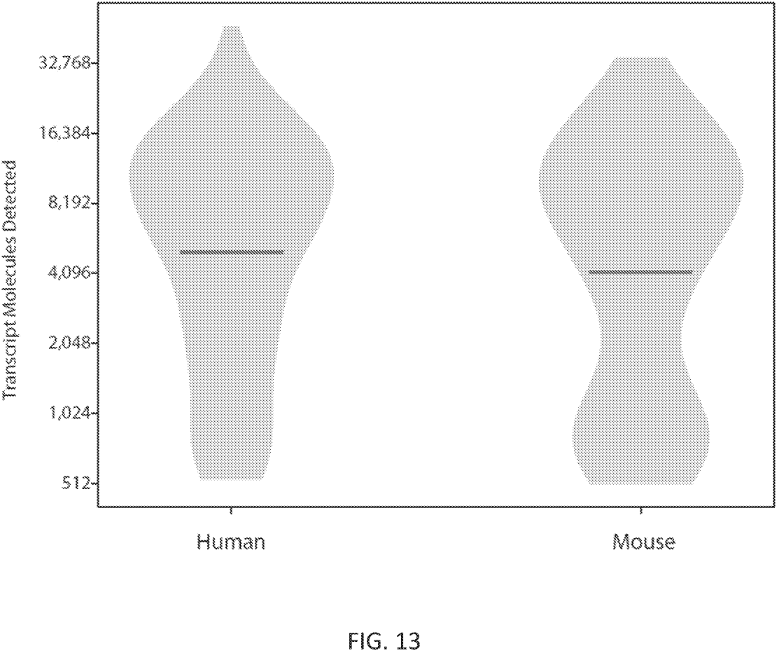

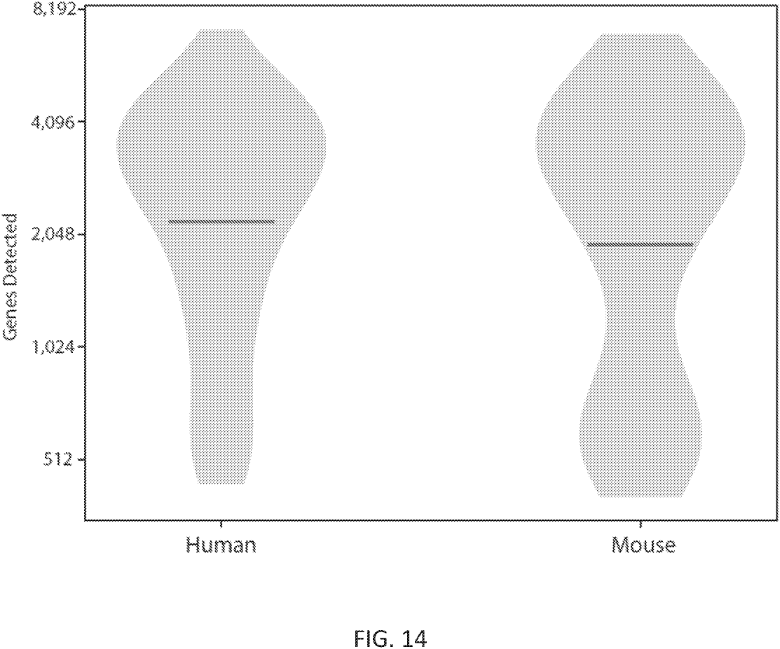

FIG. 12 is a scatter plot showing the number of human- and mouse-aligning transcript molecules for each cell-identifying barcode in a single cell RNA-seq experiment illustrating that while the majority of cell-identifying barcodes are strongly associated with one species, some are associated with both, indicating co-encapsulation of multiple cells with a bead. The methods of the present disclosure allow for the removal of multiplets from the dataset. FIG. 13 shows violin plots of the distributions of the number of transcript molecules detected per cell for cell-identifying barcodes associated with either human or mouse transcriptome annotations (where at least 70% of molecules align to either the human or mouse transcriptome) from a single cell RNA-seq experiment. FIG. 14 shows violin plots of the distributions of the number of genes detected per cell for cell-identifying barcodes associated with either human or mouse transcriptome annotations (where at least 70% of molecules align to either the human or mouse transcriptome) from a single cell RNA-seq experiment.

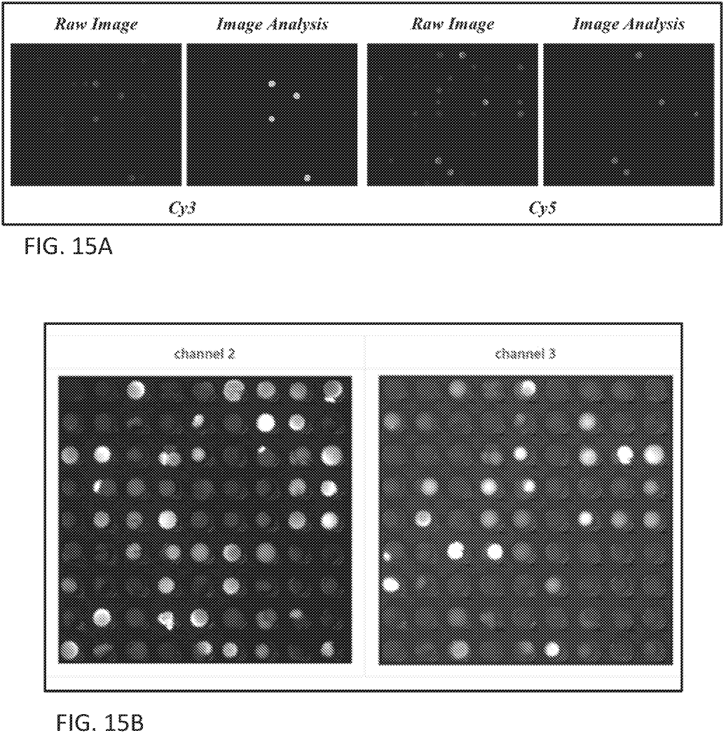

Imaging of the optical hybridization probes on the automated system is described in Example 4. FIG. 15A shows images comparing raw and analyzed fluorescence images of 8-base, Cy3-labeled and 8-base, Cy5-labeled optical probes hybridized to the complementary cell identifying optical barcode on beads present in the individual microwells of the array. FIG. 15B shows images of a cycle of fluorescence hybridization imaging in which a pooled set of 8-base, Cy5-labeled oligonucleotides and a set of 8-base, Cy3-labeled oligonucleotides were introduced into the array device loaded with beads and imaged in each of channels 2 and 3, to probe the first and second sequences, respectively, on each bead.

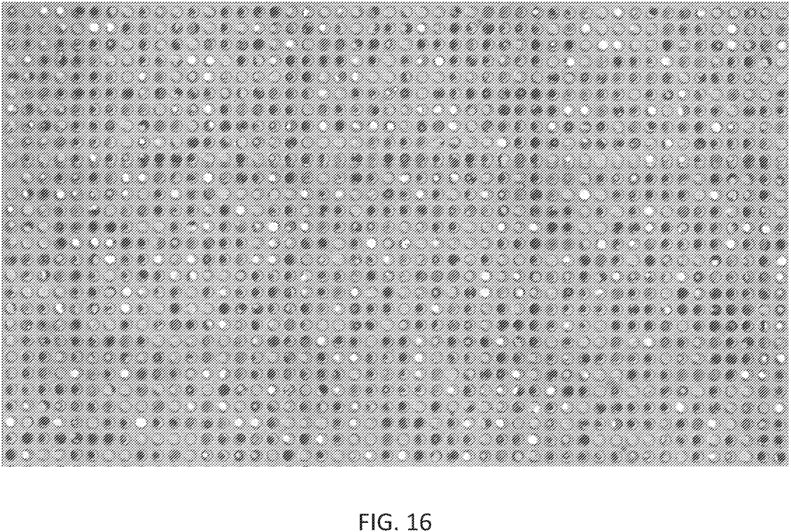

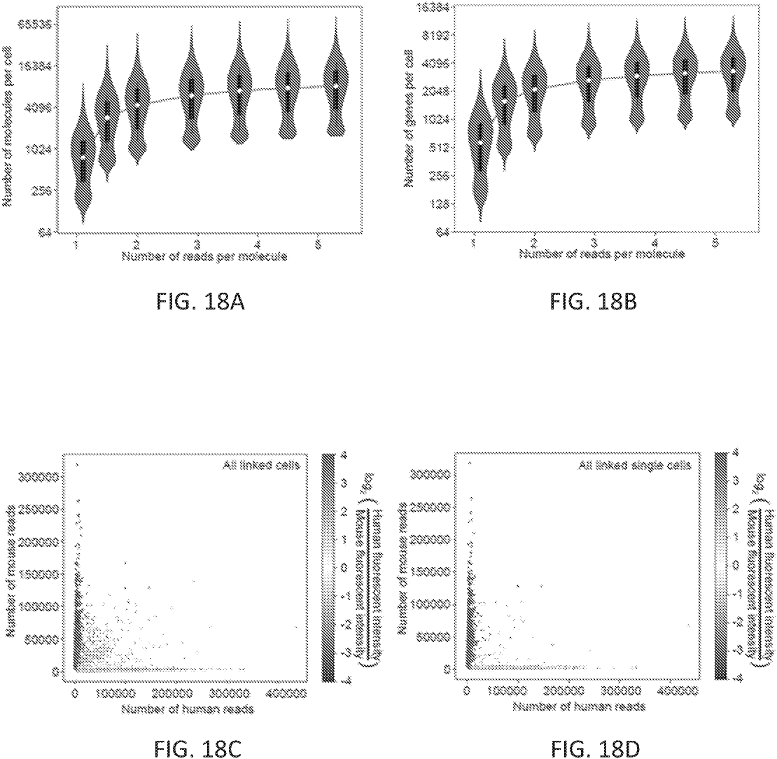

FIG. 16 is an image showing software analysis of a cycle of fluorescence hybridization imaging to identify the two barcode sequences on each bead that together form the cell identifying optical barcode sequence. A pooled set of hybridization probes consisting of 8-base, Cy5-labeled oligonucleotides and 8-base, Cy3-labeled oligonucleotides was introduced into the array device loaded with beads and imaged in each of channels 2 and 3, to probe the first and second barcode sequences, respectively, on each bead. The software analysis of this mix of pooled probes indicates the detected fluorescence as “positive” for channel 2, “positive” for channel 3, or positive for both. The automated system and methods of the present disclosure can result in high accuracy of linking of imaging and sequencing data as described in Example 6. For example, an experiment is performed to demonstrate using RNA capture beads containing cell identifying optical barcodes to link single cell phenotypic image and nucleic acid sequence data, in terms of throughput, molecular capture efficiency, and accuracy of linking imaging and sequencing data. This experiment is performed with mixed human (U87) and mouse (3T3) cells labeled with two differently colored live staining dyes. Mixed cells are loaded into the microwells and transcriptional profiles are obtained from a single experiment. At saturating sequencing depth, on average 10,245 RNA transcripts are detected from 3,548 genes per cell (FIGS. 18A, 18B). To evaluate the linking accuracy, the species of each cell is identified from the color of the fluorescent label and from the species-specific alignment rate in RNA-seq (a cell with >90% of reads aligning to the transcriptome of a given species is considered species-specific), and the consistency of the two cell species calls is examined. In the 4,145 scRNA-seq profiles that are successfully linked with imaging data, a class-balanced linking accuracy of 99.2% (0.8% error rate) is obtained, with 98.8% of human cells and 99.6% of mouse cells agreeing with the species calls from two-color imaging (FIG. 18C). In addition, multiplets are confidently removed by manually identifying mixed-species and single-species multiplets from the two-color cell images. By comparing image-based and sequencing-based mixed-species multiplets, a multiplet detection sensitivity of 68.8% and a specificity of 97.0% is obtained. A large portion of transcriptional profiles with low purity are removed (FIG. 18D). Since high linking accuracy is confirmed, it is suspected that the mixed-species multiplets detected by sequencing but not imaging are because of the imperfections in scRNA-seq data, which serves as the ground truth.

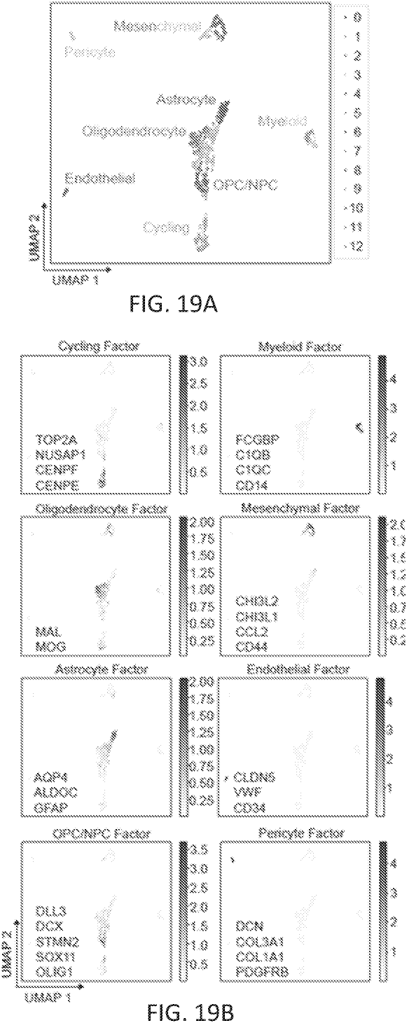

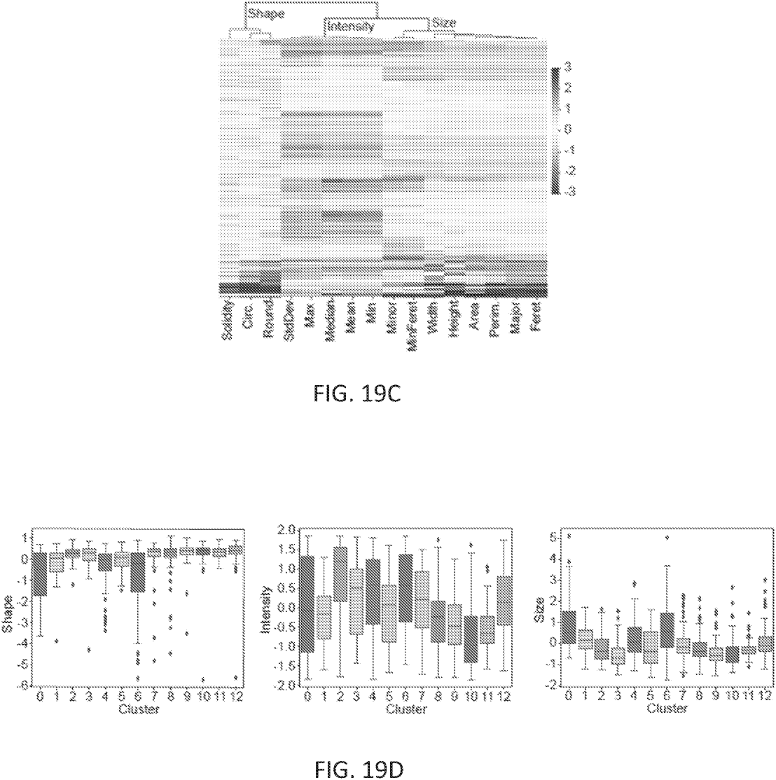

The automated system and methods of the present disclosure can be used for identifying a correspondence between single cell optical phenotypes and cell type, lineage, or clone. For example, identification of relationships between imaging features and lineage identities of malignantly transformed glioblastoma (GBM) cells is described in Example 7. To demonstrate collection of paired optical and transcriptional phenotypes from human tissue samples using the cell identifying optical barcodes described herein, an experiment is performed on cells dissociated from a human GBM surgical sample and labeled with calcein AM, a fluorogenic dye that reports esterase activity. 1,954 scRNA-seq profiles are obtained and 1,110 of them linked to live cell images. Cell multiplets are removed based on imaging analysis. A large population of cells is identified with amplification of chromosome 7 and loss of chromosome 10, two commonly co-occurring aneuploidies that are pervasive in GBM, based on the gene expression. Key gene signatures that define the population are identified by computational analysis. All of the major cell types are recovered that have been previously reported from scRNA-seq of GBM including myeloid cells, endothelial cells, pericytes, malignant-transformed astrocyte-like cells, mesenchymal-like cells, oligodendrocyte-progenitor-like/neuroblast-progenitor-like cells (OPC/NPC) and cycling cells (FIGS. 19A, 19B). Sixteen imaging features are measured from cell images and those features grouped into three categories of cell size, shape and calcein AM intensity using unsupervised hierarchical clustering (FIG. 19C) to create three imaging-based meta-features. By linking the meta-features to scRNA-seq cell types, myeloid cells (clusters 7 and 12) are found to be relatively round and small with high esterase activity; endothelial cells are large and less round as expected, and have intermediate esterase activity; and pericytes have intermediate shape, size and intensity (FIG. 19D).

Malignant cells in GBM can resemble multiple neural lineages and exhibit a mesenchymal phenotype. Because malignant GBM cells are known to be highly plastic and undergo differentiation and de-differentiation, a diffusion map is used to visualize their lineage relationships. Malignant cells are selected based on aneuploidy as described above, the dimensionality of malignant cell gene expression is reduced, and the factorized data are visualized with a diffusion map, which reveals two major branches. One branch consists of astrocyte-like cells and terminates with mesenchymal-like cells, while the other branch consists of OPC/NPC cells and cycling cells. This is consistent with previously published studies showing that astrocyte-like and mesenchymal glioma cells are significantly more quiescent than OPC-like glioma cells.

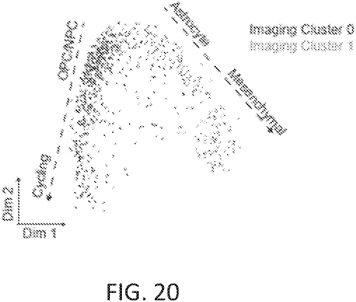

To explore how imaging features of malignant cells are related to the two major cellular lineages, it is asked whether unsupervised clustering of cellular imaging features would correspond to the two major lineages observed in scRNA-seq. Malignant cells are clustered by the three imaging meta-features described above using hierarchical clustering, and two major cellular imaging clusters are identified. By plotting two imaging clusters on the diffusion map embedding of the malignant cells, it is found that cells with round shape, low intensity and small size (imaging cluster 0) are enriched in the OPC/NPC-cycling branch, and cells with rough shape, high intensity and large size (imaging cluster 1) are enriched in the astrocyte-mesenchymal branch (FIG. 20). This finding is further supported by differential expression analysis comparing expression profiles of cells in the two imaging clusters. As expected, markers of OPC/NPCs (MAP2, OLIG1, DLL3) and cycling cells (CDK6) are significantly enriched in imaging cluster 0, while markers of astrocyte-like cells (APOE, GFAP, GJA1, AQP4, ALDOC) and mesenchymal cells (CHl3L1, CD44, CHl3L2, CCL2) are significantly enriched in imaging cluster 1. Therefore, there is a clear correspondence between the major gene expression and basic imaging features for the malignantly transformed cells in this tumor.

An example method is provided for identifying a correspondence between single cell optical phenotypes and cell type, lineage, or clone. The method includes: initializing a system, the system comprising: an instrument assembly comprising a fluidics subsystem, a thermal subsystem, and an imaging subsystem, wherein the imaging subsystem comprises a stage configured for holding a microwell array; a control subsystem coupled to the instrument assembly, the control subsystem comprising at least one processor and memory; and using the control subsystem for performing operations. The operations including flowing, using the fluidics subsystem, a plurality of cells onto the microwell array, wherein a subset of the cells reside as single cells in the microwells; obtaining, for each position of a plurality of positions in the microwell array, one or more first images at the position using the imaging subsystem and measuring one or more of a cell optical phenotypic feature; flowing, using the fluidics subsystem, a plurality of microbeads having a cell identifying optical barcode sequence and an RNA binding sequence onto the microwell array, wherein a subset of the beads reside as a single cell-bead pair in the microwells; flowing, using the fluidics subsystem, a cell lysis buffer and one or more reagents for RNA library preparation onto the microwell array; flowing, using the fluidics subsystem, a first of N pools of a plurality of optical hybridization probes onto the microwell array and hybridizing the probes to the beads located therein having a complementary nucleotide sequence in the cell identifying optical barcode sequence; obtaining, for each position of the plurality of positions, one or more second images to quantify a fluorescent intensity at the position using the imaging subsystem, each of the one or more second images used to create a binary code depicting a match or a lack of a match between at least one of the optical hybridization probes and the cell identifying optical barcodes; repeating the flowing and hybridizing step and obtaining of the one or more second images step for each of the N pools of probes; determining, by mapping the binary code for each of the N pools of probes to the cell identifying barcode sequence, for each position of the plurality of positions, the cell identifying optical barcode for the position, and storing a data association between the cell identifying optical barcode for the position and the first image at the position; storing, for each position of the plurality of positions, after receiving nucleic acid sequencing data for each cell identifying optical barcode, a data association between the nucleic acid sequencing data, the cell identifying optical barcode, and the first image associated with the cell identifying optical barcode. The method includes generating a representation of the relationship between the one or more cell optical phenotypic features and the nucleic acid sequencing data associated with each of the first images, wherein a correlation between the single cell phenotypic features and the associated sequencing data identifies a correspondence between single cell optical phenotypes and cell type, lineage, or clone based on transcriptomics of that single cell.

In one example, the cell optical phenotypic feature is one or more of area, mean intensity, standard deviation of intensity, minimum intensity, maximum intensity, median intensity, perimeter, width, height, major axis, minor axis, circularity, Feret’s diameter, minimum Feret’s diameter, roundness, or solidity; however, the method is not limited to these cell optical phenotypic features. One advantage of this method is that a broad repertoire of cell optical phenotypic features can be measured including intracellular in addition to surface features. This contrasts with FACS, in which only changes expressed on the surface of cells can be identified.

The cell optical phenotypic feature can be derived from bright-field, dark field, fluorescence, luminescence, Raman, or scattering microscopy or other microscopies, as is understood to those of skill in the art.

In the method of identifying a correspondence between single cell optical phenotypes and cell type, lineage, or clone, the cells can comprise a tissue, a tumor, a cell culture, or any type of a bodily fluid, including, but not limited to, a blood sample, a urine sample, or a saliva sample.

In the method, the cells can be human, mammal, or animal cells. In one example, the cells are immune cells, T cells, B cells, stromal cells, stem cells, neural cells, or tumor cells.

In one example of the method of identifying a correspondence between single cell optical phenotypes and cell type, lineage, or clone, the cells are immune cells and the optical phenotypic features measured includes immunophenotyping features, such as is known to those of skill in the art to characterize the immune phenotype of an immune cell type.

In another example of the method of identifying a correspondence between single cell optical phenotypes and cell type, lineage, or clone, the cells used in the method are cells that have been subject to genetic modification. By measuring one or more cell optical phenotypic features for the gene edited cells, the goal is to identify a correspondence between the optical phenotypic features and the cell clones that either have or do not have the genetic modification. Once this correspondence is identified, the desired cell clones either positive or negative for the genetic modification can be identified by optical methods rather than requiring more expensive gene sequencing. This has applications for cells for immunotherapy as well as others. In one example, the cells that have been subject to genetic modification are stem cells, immune cells, T cells, or B cells.

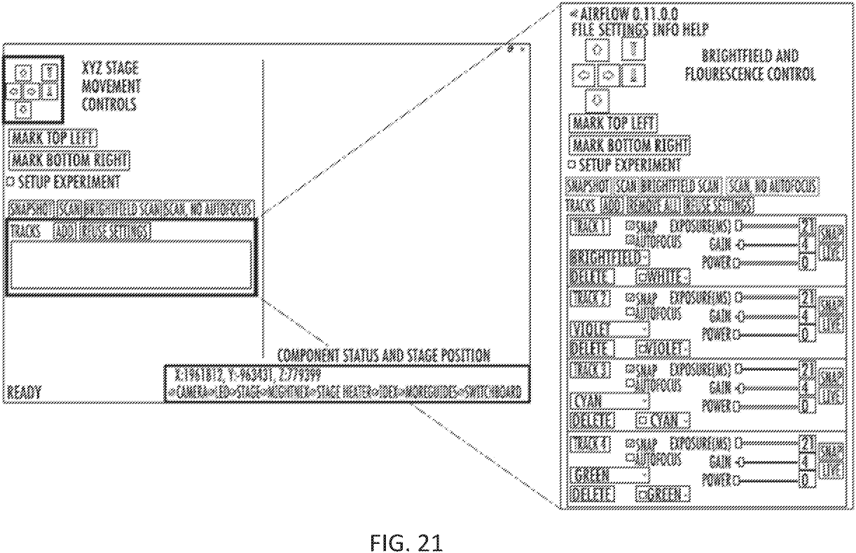

FIG. 21 includes a screenshot of an example GUI for controlling various aspects of the process, in particular, setting the parameters for a bright-field and multi-channel fluorescence scan of the microwell array. FIG. 21 shows various user interface controls for controlling the bright-field and fluorescence channels of an experiment. The GUI also includes user interface controls for manually moving the XY stage and autofocus motor.

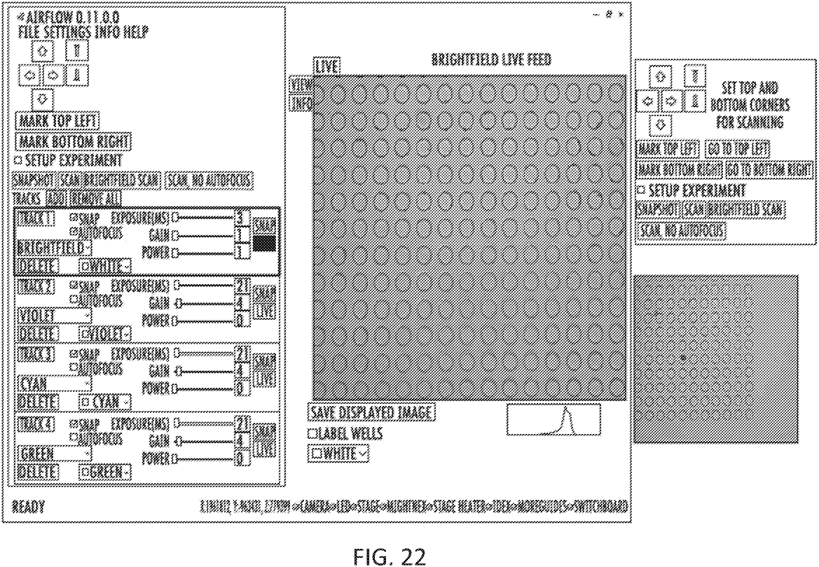

FIG. 22 is another screenshot of an example GUI for viewing live bright-field images of the microwell array to set the imaging parameters for the bright-field channel of the scan. FIG. 22 shows an example live view, i.e., a view of the microwell array from the imaging system. Using the user interface controls, a user can look at live images, e.g., to see if the focus is appropriate, or to mark the top left and bottom right corners of the microwell array to set boundaries for a scan.

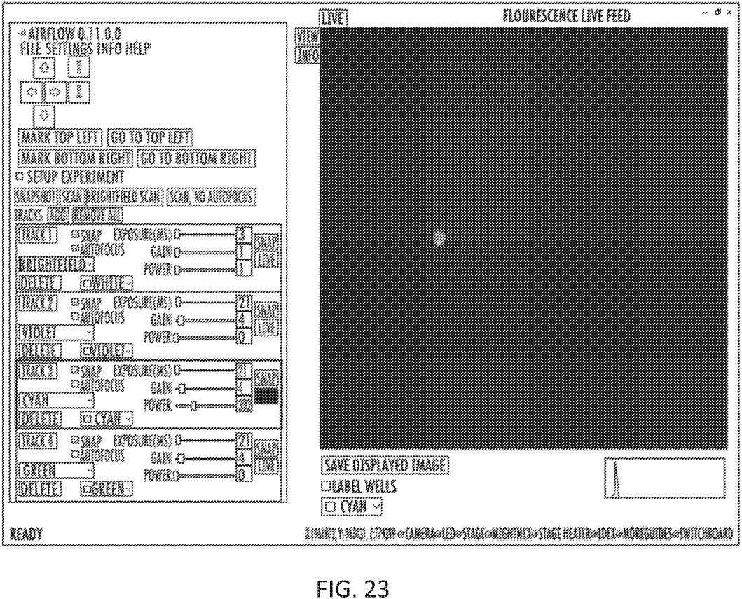

FIG. 23 is screenshot of an example GUI for viewing live images of the microwell array in one of the fluorescence channels to set the imaging parameters for that channel of the scan. In the example shown in FIG. 23, the GUI shows a fluorescence live feed, e.g., to observe a cell or a bead.

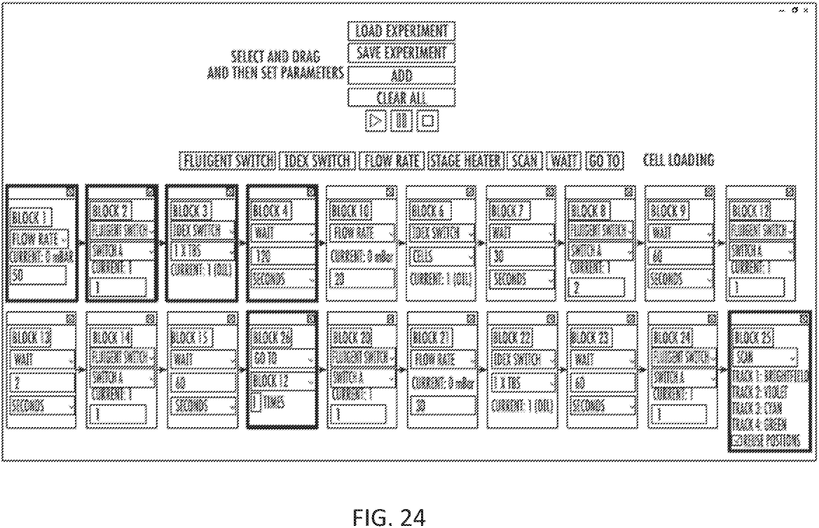

FIG. 24 is a screenshot of an example GUI for setting various parameters for a cell loading operation. The GUI includes various user interface elements for specifying properties of an experiment and initiating a scan of a microwell array.

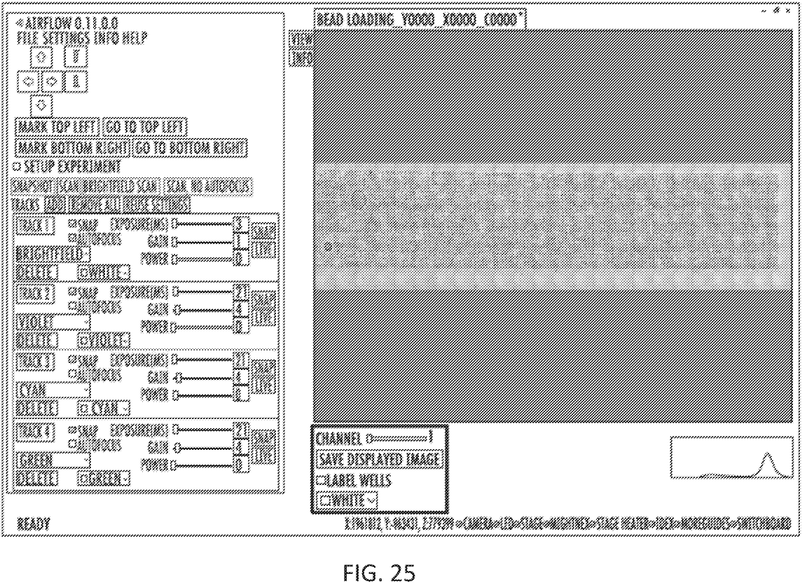

FIG. 25 is a screenshot of an example GUI for viewing bright-field imaging results of a scan of the microwell array. The example shown in FIG. 25 shows a mosaic of different images stitched together into a single image.

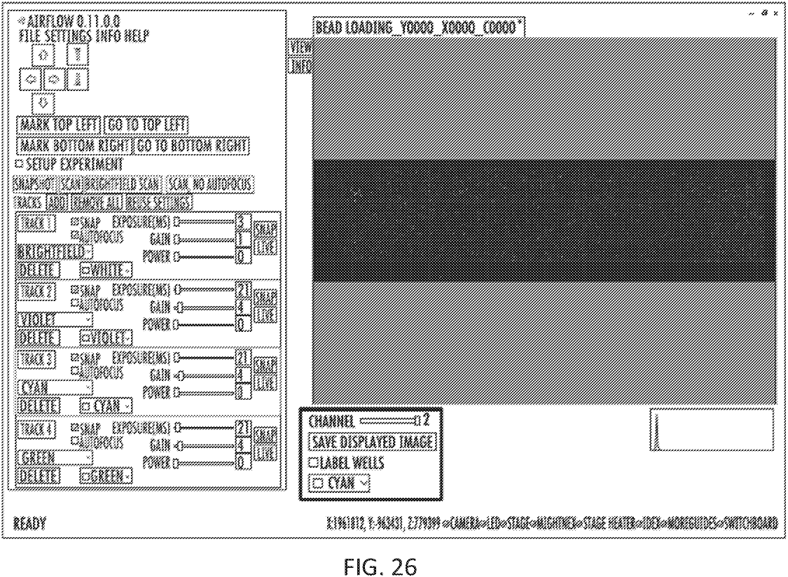

FIG. 26 is a screenshot of another GUI for viewing fluorescence imaging results of a scan of the microwell array. The user interface controls can be used to specify the viewing parameters.

In one example of an automated system of the present disclosure, the system is used for associating single cell imaging with unique optical barcode readout, and preparation of sequencing libraries other than RNA libraries. For example, the system comprising: an instrument assembly comprising a fluidics subsystem, a thermal subsystem, and an imaging subsystem, wherein the imaging subsystem comprises a stage configured for holding a microwell array; a control subsystem coupled to the instrument assembly, the control subsystem comprising at least one processor and memory, the control subsystem configured for performing operations comprising: flowing, using the fluidics subsystem, a plurality of cells onto the microwell array, wherein a subset of the cells reside as single cells in the microwells; obtaining, for each position of a plurality of positions in the microwell array, one or more first images of the cell at the position using the imaging subsystem; flowing, using the fluidics subsystem, a plurality of microbeads having a cell identifying optical barcode sequence and a primer sequence to capture cellular nucleic acid onto the microwell array, wherein a subset of the beads reside as a single cell-bead pair in the microwells; flowing, using the fluidics subsystem, a cell lysis buffer and one or more reagents for sequencing library preparation onto the microwell array; flowing, using the fluidics subsystem, a first of N pools of a plurality of optical hybridization probes onto the microwell array and hybridizing the probes to the beads located therein having a complementary nucleotide sequence in the cell identifying optical barcode sequence; obtaining, for each position of the plurality of positions, one or more second images to quantify a fluorescent intensity at the position using the imaging subsystem, each of the one or more second images used to create a binary code depicting a match or a lack of a match between at least one of the optical hybridization probes and the cell identifying optical barcodes; repeating the flowing and hybridizing step and obtaining of the one or more second images step for each of the N pools of probes; and determining, by mapping the binary code for each of the N pools of probes to the cell identifying barcode sequence, for each position of the plurality of positions, the cell identifying optical barcode for the position and storing a data association between the cell identifying optical barcode for the position and the first image at the position.

In this example of the automated system, the primer sequence designed to capture cellular nucleic acid can be an oligo(dT) to capture RNA, mRNA, and non-coding RNA; a random sequence to capture any DNA or RNA; or a specific sequence targeted to a DNA loci or an RNA transcript.

In one example, the automated system of the present disclosure can be used in a method for associating single cell imaging data with nucleic acid sequencing data, rather than for just RNA transcriptomics. For example, the method comprising: initializing a system, the system comprising: an instrument assembly comprising a fluidics subsystem, a thermal subsystem, and an imaging subsystem, wherein the imaging subsystem comprises a stage configured for holding a microwell array; a control subsystem coupled to the instrument assembly, the control subsystem comprising at least one processor and memory; and using the control subsystem for performing operations comprising: flowing, using the fluidics subsystem, a plurality of cells onto the microwell array, wherein a subset of the cells reside as single cells in the microwells; obtaining, for each position of a plurality of positions in a microwell array, one or more first images at the position using the imaging subsystem; flowing, using the fluidics subsystem, a plurality of microbeads having a cell identifying optical barcode sequence and a primer sequence to capture cellular nucleic acid onto the microwell array, wherein a subset of the beads reside as a single cell-bead pair in the microwells; flowing, using the fluidics subsystem, a cell lysis buffer and one or more reagents for sequencing library preparation onto the microwell array; flowing, using the fluidics subsystem, a first of N pools of a plurality of optical hybridization probes onto the microwell array and hybridizing the probes to the beads located therein having a complementary nucleotide sequence in the cell identifying optical barcode sequence; obtaining, for each position of the plurality of positions, one or more second images to quantify a fluorescent intensity at the position using the imaging subsystem, each of the one or more second images used to create a binary code depicting a match or a lack of a match between at least one of the optical hybridization probes and the cell identifying optical barcodes; repeating the flowing and hybridizing step and obtaining of the one or more second images step for each of the N pools of probes; determining, by mapping the binary code for each of the N pools of probes to the cell identifying barcode sequence, for each position of the plurality of positions, the cell identifying optical barcode for the position and storing a data association between the cell identifying optical barcode for the position and the first image at the position; and storing, for each position of the plurality of positions, after receiving nucleic acid sequencing data for each cell identifying optical barcode, a data association between the nucleic acid sequencing data, the cell identifying optical barcode, and the first image associated with the cell identifying optical barcode wherein the single cell imaging data is thereby associated with the nucleic acid sequence for that cell.

In the example of this automated method, the primer sequence can an oligo(dT) to capture RNA, mRNA, and non-coding RNA; a random sequence to capture any DNA or RNA; or a specific sequence targeted to a DNA loci or an RNA transcript.

In one example, the automated system of the present disclosure can be used in a method for identifying a correspondence between single cell optical phenotypes and cell type, lineage, or clone, comprising: initializing a system, the system comprising: an instrument assembly comprising a fluidics subsystem, a thermal subsystem, and an imaging subsystem, wherein the imaging subsystem comprises a stage configured for holding a microwell array; a control subsystem coupled to the instrument assembly, the control subsystem comprising at least one processor and memory; using the control subsystem for performing operations comprising: flowing, using the fluidics subsystem, a plurality of cells onto the microwell array, wherein a subset of the cells reside as single cells in the microwells; obtaining, for each position of a plurality of positions in the microwell array, one or more first images at the position using the imaging subsystem and measuring one or more of a cell optical phenotypic feature; flowing, using the fluidics subsystem, a plurality of microbeads having a cell identifying optical barcode sequence and a primer sequence to bind cellular nucleic acid onto the microwell array, wherein a subset of the beads reside as a single cell-bead pair in the microwells; flowing, using the fluidics subsystem, a cell lysis buffer and one or more reagents for sequencing library preparation onto the microwell array; flowing, using the fluidics subsystem, a first of N pools of a plurality of optical hybridization probes onto the microwell array and hybridizing the probes to the beads located therein having a complementary nucleotide sequence in the cell identifying optical barcode sequence; obtaining, for each position of the plurality of positions, one or more second images to quantify a fluorescent intensity at the position using the imaging subsystem, each of the one or more second images used to create a binary code depicting a match or a lack of a match between at least one of the optical hybridization probes and the cell identifying optical barcodes; repeating the flowing and hybridizing step and obtaining of the one or more second images step for each of the N pools of probes; determining, by mapping the binary code for each of the N pools of probes to the cell identifying barcode sequence, for each position of the plurality of positions, the cell identifying optical barcode for the position, and storing a data association between the cell identifying optical barcode for the position and the first image at the position; storing, for each position of the plurality of positions, after receiving nucleic acid sequencing data for each cell identifying optical barcode, a data association between the nucleic acid sequencing data, the cell identifying optical barcode, and the first image associated with the cell identifying optical barcode. The method includes generating a representation of the relationship between the one or more cell optical phenotypic features and the nucleic acid sequencing data associated with each of the first images, wherein a correlation between the single cell phenotypic features and the associated sequencing data identifies a correspondence between single cell optical phenotypes and cell type, lineage, or clone based on nucleic acid sequence of that single cell.

In the example method, the primer sequence can be an oligo(dT) to capture RNA, mRNA, and non-coding RNA; a random sequence to capture any DNA or RNA; or a specific sequence targeted to a DNA loci or an RNA transcript.

Accordingly, while the methods and systems have been described in reference to specific embodiments, features, and illustrative embodiments, it will be appreciated that the utility of the subject matter is not thus limited, but rather extends to and encompasses numerous other variations, modifications and alternative embodiments, as will suggest themselves to those of ordinary skill in the field of the present subject matter, based on the disclosure herein.

Various combinations and sub-combinations of the structures and features described herein are contemplated and will be apparent to a skilled person having knowledge of this disclosure. Any of the various features and elements as disclosed herein may be combined with one or more other disclosed features and elements unless indicated to the contrary herein. Correspondingly, the subject matter as hereinafter claimed is intended to be broadly construed and interpreted, as including all such variations, modifications and alternative embodiments, within its scope and including equivalents of the claims.

### EXAMPLES

### Example 1

### Single Cell RNA-Seg on the Automated System

Device preparation. A microwell array device was fabricated from polydimethylsiloxane (PDMS), a commonly used elastomeric polymer, and stored in a humid chamber in wash buffer (20 mM Tris-HCl pH 7.9, 50 mM NaCl, 0.1% Tween-20) one day before use.

Cell preparation. Five different experiments were performed in which 4 of the experiments involved mixed mouse (3T3)/human (U87) cells and one was with U87 human cells alone. Cells were dissociated into single cell suspensions using 0.25% Trypsin-EDTA (Life Technologies, cat# 25200-072); human U87 cells were stained with Calcein AM (ThermoFisher Scientific, cat# C3100MP) and mouse 3T3 cells were stained with Calcein red-orange (ThermoFisher Scientific, cat# C34851) in 1X TBS at 37° C. for 15 minutes. The U87 and 3T3 cells were mixed at 1:1 ratio with a final total cell concentration 1000 cells/µl.

Initialize system. The microwell array device was inserted into the instrument assembly and the automated system was configured for automated cell and bead loading followed by single cell RNA sequencing library preparation. The single cell suspension was loaded into the cell loading reservoir. The beads (Chemgenes Drop-SEQ beads) were added to the bead loading reservoir. Single cell RNA-Seq library preparation reagents were loaded into the reagent reservoirs and the reagent reservoirs were attached to the instrument assembly.

The following steps were performed on the automated system:

Cell loading. After flowing Tris-buffered saline (TBS) through the device, single cells were loaded into individual microwells of the device at a density of approximately 10% (see FIG. 8A).

Cellular imaging. The cell-loaded microwell device was scanned under the bright-field and fluorescence channels (FIG. 8A). Bright-field images were taken using an LED light source and wide-field 10x 0.3 NA objective. Fluorescence images were taken using LED light source, quad-band filter set, wide-field 10x 0.3 NA objective with 470 nm (GFP channel) and 555 nm (TRITC channel) excitation for Calcein AM and Calcein red-orange, respectively.

Imaging based multiplets identification. Two-color live staining fluorescence images were merged with Calcein AM signal in green and Calcein red-orange signal in magenta. Each well was automatically examined within the smallest bounding square. Wells with mixed-species cells were determined as having at least one green object and one magenta object; wells with a single cell were determined as having only one green object or one magenta object.

Bead loading and imaging. After washing the microwell device with TBS, beads were loaded into individual microwells of the device to an approximate density of 80% as confirmed by imaging (FIG. 8A).

Cell lysis and imaging. After washing the microwell array device with TBS, lysis buffer (1% 2-Mercaptoethanol (Fisher Scientific, cat# BP176-100), 99% Buffer TCL (Qiagen, cat# 1031576)) followed by perfluorinated oil (Sigma-Aldrich, cat# F3556-25ML) was flowed into the device and incubated at 50° C. for 20 minutes to promote cell lysis. The device was imaged as a quality control step to assess the extent of cell lysis (FIG. 8A). After lysis, the temperature of the device was held at 25° C. for 90 minutes to promote RNA capture onto the beads. Wash buffer supplemented with RNase inhibitor (0.02 U/µL SUPERaselN (Thermo Fisher Scientific, cat# AM2696) in wash buffer) was flushed through the device to unseal the microwells and remove any uncaptured RNA molecules. The device was imaged again as a quality check to ensure sufficient removal of fluorescent cell lysate (see FIG. 8B).

Image analysis. Lysis was confirmed using ImageJ to analyze images. To identify microwells, the difference was taken between the background and the bright-field image, then the threshold calculated using Otsu’s method (https://doi.org/10.1109/TSMC.1979.4310076). The threshold was used to generate a binary image, which was then dilated, and holes were filled. The binary objects were identified to create a mask of the wells to measure cell loading and lysis efficiency. After cell loading, the average fluorescence intensities of microwells in the live staining images were measured. Average intensity values followed a bimodal distribution, with the higher intensity population corresponding to microwells that contain cells. After cell lysis, the fluorescence intensity of the microwell device was measured and the lysis efficiency was calculated for wells that originally contained a cell. FIG. 10A shows a binary image of segmented and labeled microwells. FIG. 10B shows a bright-field image of cells in microwells. FIG. 10C shows a fluorescent image of live-stained cells. FIG. 10D shows a fluorescent image of the microwells in FIG. 10C after cell lysis.

Reverse transcription. Reverse transcription mixture (1X Maxima RT buffer, 1 mM dNTPs, 1 U/µL SUPERaselN, 2.5 µM template switch oligo, 10 U/µL Maxima H Minus reverse transcriptase (Thermo Fisher Scientific, cat# EP0752), 0.1% Tween-20) was flowed into the device followed by an incubation at 25° C. for 30 minutes and then at 42° C. for 90 minutes. Wash buffer supplemented with RNase inhibitor was flushed through the device.

The microwell device was removed from the instrument assembly and Exonuclease I reaction mixture (1X Exo-I buffer, 1 U/µL Exo-I (New England Biolabs, cat# M0293L)) was flowed through the device followed by an incubation at 37° C. for 45 minutes. TE/TW buffer (10 mM Tris pH 8.0, 1 mM EDTA, 0.01% Tween-20) was flushed through the device. The beads were collected and pooled for sequencing. FIG. 8B shows graphs of capillary and gel electrophoresis analysis of the bead-free PCR product extracted from beads subjected to the on-device workflow and negative control beads (i.e., Drop-SEQ beads that were not subjected to on-device reverse transcription).

### PCR and Sequencing Performed Off the Automated System

The pooled beads were washed sequentially with TE/SDS buffer (10 mM Tris-HCl, 1 mM EDTA, 0.5% SDS), TE/TW buffer, and nuclease-free water. cDNA amplification was performed in 50 µL PCR solution (1X Hifi Hot Start Ready mix (Kapa Biosystems, cat# KK2601), 1 µM SMRTpcr primer (Table EV5)), with 14 amplification cycles (95° C. 3 min, 4 cycles of (98° C. 20 s, 65° C. 45 s, 72° C. 3 min), 10 cycles of (98° C. 20 s, 67° C. 20 s, 72° C. 3 min), 72° C. 5 min) on a thermocycler. PCR product was purified using AMPure paramagnetic beads (Beckman, cat# A63881) with a bead-to-sample volume ratio of 0.6:1. Purified cDNA was then tagmented and amplified using the Nextera kit for in vitro transposition (Illumina, FC-131-1024). 0.8 ng cDNA was used as input per reaction. A unique i7 index primer was used to barcode the library. The i5 index primer was replaced by a universal P5 primer for the selective amplification of 5′ end of cDNA (corresponding to the 3′ end of RNA). Two rounds of SPRI paramagnetic bead-based purification with a bead-to-sample volume ratio of 0.6:1 and 1:1 were performed sequentially on the Nextera PCR product to obtain a sequencing-ready library. 20% PhiX library (Illumina, FC-131-1024) was spiked-in before sequencing on an Illumina NextSeq 500 with a 26-cycle read 1, 58 cycle read 2, and 8 cycle index read. A custom sequencing primer was used for read 1.

The sequencing data resulting from the5 experiments described above is shown is Table 1. The data show the automated system can produce high purity cDNA libraries from multiple cell types.

TABLE 1

| Sample | Number of Cells | Ave. Counts/Cell | Purity (%) | Multiplet Rate (%) |
|---|---|---|---|---|
| 1 | 790 | 5,600 | N.D. | N.D. |
| 2 | 270 | 3,200 | 94 | 18 |
| 3 | 588 | 3,700 | 96 | 23 |
| 4 | 355 | 6,300 | 98 | 34 |
| 5 | 347 | 4,700 | 89 | 26 |

Sub-sampling analysis. To analyze the saturation behavior and sensitivity of scRNA-seq data, the aligned reads were randomly sub-sampled and re-processed with the scRNA-seq analysis. Two statistics are then calculated, molecules per cell and genes per cell, based on the cells that are discovered from the total reads.

Validation Data. Additional data validating the sequencing results from the mixed species experiments on the automated system are shown in FIGS. 12-14.

FIG. 12 is a scatter plot showing the number of human- and mouse-aligning transcript molecules for each cell identifying barcode from one of the mixed species experiments described above. This plot illustrates that while the majority of cell identifying barcodes are strongly associated with one species, some are associated with both, indicating co-encapsulation of multiple cells with a bead.

FIG. 13 shows violin plots of the distributions of the number of transcript molecules detected per cell for cell identifying barcodes associated with either human or mouse transcriptome annotations (where at least 70% of molecules align to either the human or mouse transcriptome) from one of the mixed species experiments described above.

FIG. 14 shows violin plots of the distributions of the number of genes detected per cell for cell identifying barcodes associated with either human or mouse transcriptome annotations (where at least 70% of molecules align to either the human or mouse transcriptome) from one of the mixed species experiments described above.

### Example 2

### Construction of Beads Having Cell-Identifying Optical Barcode Sequences

8-nt cell barcode sequences were designed using an R package ‘DNAbarcodes’ with following criteria: sequences were at least 3 Levenshtein distance from each other; sequences that contain homopolymers longer than 2 nucleotides, with GC content <40% or >60%, or perfectly self-complementary sequences were removed. Sequences were further selected based on less secondary structure formation.

The bead design is illustrated in FIG. 11A. Bead synthesis was performed by Chemgenes Corp (Wilmington, MA) as illustrated in FIG. 11B. Toyopearl HW-65S resin (~30 micron mean particle diameter) (Tosoh Biosciences, cat# 19815, Tosoh Bioscience) with a flexible-chain linker was used as a solid support for reverse-direction phosphoramidite synthesis. Beads were synthesized with sequence ‘TTTTTTTAAGCAGTGGTATCAACGCAGAGTACNN’ at 50 micromole scale, split into 96 parts to add one of the “S” cell barcode sequences, pooled together to add ‘NN’, split into 96 parts to add one of the “Q” cell barcode sequences, and pooled together to add ‘NNNN’ and 30 T’s.

### Example 3

### Labeling and Generation of Optical Hybridization Probe Pools for Optical Decoding

192 oligonucleotides that are complementary to the 8-nt cell barcodes with 3′-amino modifications were synthesized and purified (Sigma-Aldrich), then resuspended in water at 200 µM. To generate probe mixtures corresponding to each bit in the binary code, oligonucleotides labeled with ‘1’ were taken (see FIG. 11C), pooled and resuspended in 0.1 M sodium tetraborate (pH 8.5) coupling buffer at a final concentration of 22 µM with 0.6 µg/µL reactive fluorophore. Sulfo-CY5 NHS ester (Lumiprobe, cat# 21320) was coupled with S oligo pools, and Sulfo-CY3 NHS ester (Lumiprobe, cat# 23320) was coupled with Q oligo pools overnight at room temperature. Excess fluorophores were removed and oligos were recovered by ethanol precipitation (80% Ethanol, 0.06 M NaCl, 6 µg/mL glycogen). The concentration of probes was quantified using a NanoDrop (Thermo Scientific). Probe pools were diluted such that each probe had a final concentration of ~20 nM, and the two, distinctly labeled probe pools are mixed together for each binary code bit prior to use.

### Example 4

### Imaging of Optical Hybridization Probes on the Automated System

The automated system steps shown in FIG. 6B of loading the optical hybridization probes, imaging, and removing the probes were validated as follows. DROP SEQ beads (Chemgenes) were loaded into the microwell array as described above in Example 1. A wash was then performed by flowing imaging buffer (2xSSC, 0.1% Tween-20) through the device. The device was scanned in the bright-field, Cy3 and Cy5 emission channels. Fluorescence images were acquired using an LED light source (Lumencor, AURA III, 390/22 nm, 475/28 nm, 555/28 nm, 635/22 nm), quad-band filter set (Semrock, LED-DA/FI/TR/Cy5-B-000), wide-field 10x objective (Olympus, UPLFLN10X2) and 555 nm and 649 nm excitation for Cy3 and Cy5, respectively. One or a pool of hybridization probes in imaging buffer at a concentration of 20 nM was flowed into the device and incubated for 10 minutes. A wash was performed to remove non-hybridized probes by flowing imaging buffer through the device. The device was scanned in the bright-field, Cy3 and Cy5 emission channels. After imaging, melting buffer was flowed into the device and incubated for 10 minutes to remove the hybridized probes. These steps are repeated one or more times, with one or more single or pooled probes. Upon completion, the device was washed by flowing imaging buffer.

FIG. 15A shows images comparing raw and analyzed fluorescence images of 8-base, Cy3-labeled and 8-base, Cy5-labeled optical probes hybridized to the complementary cell identifying optical barcode on beads present in the individual microwells of the array in the automated system of the present disclosure.

FIG. 15B shows images of a cycle of fluorescence hybridization imaging in which a pooled set of 8-base, Cy5-labeled oligonucleotides and a set of 8-base, Cy3-labeled oligonucleotides were introduced into the array device loaded with beads and imaged in each of channels 2 and 3, to probe the first and second sequences, respectively, on each bead.

FIG. 16 is an image showing software analysis of a cycle of fluorescence hybridization imaging to identify the two barcode sequences on each bead that together form the cell identifying optical barcode sequence. A pooled set of hybridization probes consisting of 8-base, Cy5-labeled oligonucleotides and 8-base, Cy3-labeled oligonucleotides was introduced into the array device loaded with beads and imaged in each of channels 2 and 3, to probe the first and second barcode sequences, respectively, on each bead. The software analysis of this mix of pooled probes indicates the detected fluorescence as “positive” for channel 2, “positive” for channel 3, or positive for both.

### Example 5

### Single Cell RNA-Seq and Optical Decoding Using RNA Capture Beads Having Cell Identifying Optical Barcode Sequences

In the present experiment, 96 out of 256 possible binary codes are used (see FIGS. 11A-C and Examples 2 and 3 for design and synthesis of beads and optical hybridization probes), and more importantly, the number of sequenced cell identifying optical barcodes (< 10,000 cells per experiment) is much fewer than the total 92,160 possible barcodes. Therefore, an error in optical decoding would mainly result in assigning the bead an unmappable binary code, or a cell identifying optical barcode that does not appear in the sequencing data. Both kinds of misassignments further lead to the failure of linking imaging and sequencing data sets rather than incorrect linking. Thus, a more accurate optical decoding method would give a higher fraction of linked imaging and sequencing data.

To compare the ‘bead-by-bead’ optical decoding method with the ‘cycle-by-cycle’ method, two methods are tested on two datasets.

To decode the cell identifying optical barcode sequences from imaging, a ‘cycle-by-cycle’ method is used, which calls the binary code for each bead based on the bimodal distribution of intensity values across all beads in each hybridization cycle. This method works well when the bead fluorescence intensity values of the ‘one’ state population are well separated from that of the ‘zero’ state population. However, because the beads exhibit auto-fluorescence at shorter wavelengths, the two populations are not clearly separated in the Cy3 emission channel.

To accurately decode the cell barcode sequences from imaging, a modified ‘bead-by-bead’ fluorescence intensity analysis strategy is utilized. The cell barcode sequences of each bead are determined by sorting the eight intensity values in ascending order, calculating the relative intensity change between each pair of adjacent values, establishing a threshold based on the largest relative intensity change to assign a binary code, and mapping the binary code to the actual cell barcode sequence (FIG. 17A). For those unmappable binary codes, the binary code is repeatedly reassigned based on the next largest relative intensity change until the code can be successfully mapped to a cell barcode sequence. Since this method decodes each bead independently, it can be expected to provide better results when the ‘one’ and ‘zero’ intensity states are poorly separated.

In dataset PJ070 and PJ069, 46% and 57% scRNA-seq profiles are linked with cell images using the ‘bead-by-bead’ method in comparison to only 24% and 37% using the ‘cycle-by-cycle’ method. In both datasets, at least a 20% increase is observed in the fraction of linked cells with the ‘bead-by-bead’ method (FIG. 17B), which suggests that the ‘bead-by-bead’ method is more suitable for cell identifying optical barcode sequence decoding by image analysis.

The following experiment is performed to compare optical decoding methods:

Preparation. A microwell array device is filled with wash buffer (20 mM Tris-HCl pH7.9, 50 mM NaCl, 0.1% Twe20) and stored in a humid chamber one day before use. Cell culture or tissue samples are dissociated into single cell suspension and stained with desired fluorescent dyes.

Cell loading. The pre-filled microwell array device is flushed with Tris-buffered saline (TBS). The single cell suspension is pipetted into the microwell array device. After 3-minute, un-trapped cells are then flushed out with TBS.

Cellular imaging. The cell-loaded microwell device is scanned using an automated fluorescence microscope (Nikon, Eclipse Ti2) under the bright-field and fluorescence channels. Bright-field images are taken using an RGB light source (Lumencor, Lida) and wide-field 10x 0.3 NA objective (Nikon, cat# MRH00101). Fluorescence images are taken using LED light source (Lumencor, SPECTRA X), Quad band filter set (Chroma, cat# 89402), wide-field 10x 0.3 NA objective (Nikon, cat# MRH00101) with 470 nm (GFP channel) and 555 nm (TRITC channel) excitation for Calcein AM and Calcein red-orange, respectively.

scRNA-seq (steps performed on microwell device). Beads (Chemgenes) are pipetted into the microwell device, and untrapped beads are flushed out with 1x TBS. The microwell device containing the cells and the beads is connected to the computer-controlled reagent and temperature delivery system as previously described. Lysis buffer (1% 2-Mercaptoethanol (Fisher Scientific, cat# BP176-100), 99% Buffer TCL (Qiagen, cat# 1031576) and perfluorinated oil (Sigma-Aldrich, cat# F3556-25ML) is flowed into the device followed by an incubation at 50° C. for 20 minutes to promote cell lysis, and then at 25° C. for 90 minutes for RNA capture. Wash buffer supplemented with RNase inhibitor (0.02 U/µL SUPERaselN (Thermo Fisher Scientific, cat# AM2696) in wash buffer) is flushed through the device to unseal the microwells and remove any uncaptured RNA molecules. Reverse transcription mixture (1X Maxima RT buffer, 1 mM dNTPs, 1 U/µL SUPERaselN, 2.5 µM template switch oligo, 10 U/µL Maxima H Minus reverse transcriptase (Thermo Fisher Scientific, cat# EP0752), 0.1% Tween-20) is flowed into the device followed by an incubation at 25° C. for 30 minutes and then at 42° C. for 90 minutes. Wash buffer supplemented with RNase inhibitor is flushed through the device. The device is disconnected from the automated reagent delivery system. Exonuclease I reaction mixture (1X Exo-I buffer, 1 U/µL Exo-I (New England Biolabs, cat# M0293L)) is pipetted into the device followed by an incubation at 37° C. for 45 minutes. TE/TW buffer (10 mM Tris pH 8.0, 1 mM EDTA, 0.01% Tween-20) is flushed through the device.

Optical demultiplexing methods. The microwell device containing the beads with cDNAs is connected to a computer-controlled reagent delivery and scanning system. Melting buffer (150 mM NaOH) is infused into the device and incubated for 10 minutes. The device is then washed with imaging buffer (2xSSC, 0.1% Tween-20). An automated imaging program scans the device in the bright-field, Cy3 and Cy5 emission channels. Fluorescence images are acquired using an LED light source (Lumencor, spectra x), Quad band filter set (Chroma, cat# 89402), wide-field 10x objective (Nikon, cat# MRH00101) and 555 nm and 649 nm excitation for Cy3 and Cy5, respectively. Hybridization solution (imaging buffer supplemented with probe pool A, described below) is infused into the device and incubated for 10 minutes. The device is then washed with imaging buffer. An automated imaging program scans the device in the bright-field, Cy3 and Cy5 emission channels. Repeat the previous step 7 times, with probe pool B to H. Melting buffer is infused into the device and incubates for 10 minutes. The device is then washed with imaging buffer, and then disconnected from the automated reagent delivery system.

Creation of Optical Probe Pools. To link cellular imaging with scRNA-seq from the same cell, the cell identifying optical barcode sequence on each bead in the microwell array is identified by sequential fluorescent probe hybridization. A temporal barcoding strategy is used in which each cell identifying optical barcode sequence corresponds to a unique, pre-defined 8-bit binary code (See FIGS. 11A-11B). Each bit of the binary code can be read out by one cycle of probe hybridization, where the presence or absence of a hybridized probe indicates one or zero, respectively. The two parts of the cell barcode can be decoded simultaneously using two sets of differently colored fluorescent probes. To realize this decoding scheme, a pool of fluorescent probes is generated for each cycle of hybridization. All probes that can be hybridized to the cell barcode sequence marked ‘1’ in the corresponding binary code are pooled and conjugated with fluorophores, Cy5 or Cy3 (FIG. 11C). Distinct fluorophore-conjugated probes against the two 8-nucleotide cell barcode sequences “S” and “Q” together comprising the cell identifying optical barcode are then pooled together to form the final probe pool (FIG. 11C). Thus, all possible cell barcode sequences are decoded by eight cycles of two-color probe hybridization. This approach is scalable and provides a bright signal on the bead surface because every primer contains an optically decodable barcode. Thus, the beads containing the cell identifying optical barcodes are compatible with high speed imaging, leading to high throughput.

scRNA-seq Steps Performed off Microwell Device. Perfluorinated oil is pipetted into the device containing cells and the beads to seal the microwells. The device is then cut into 10 regions. Beads from each region are extracted separated by soaking each small piece of bead-containing PDMS in 100% ethanol, vortexing, water bath sonication, and centrifugation in a 1.7 mL microcentrifuge tube. PDMS is then removed by tweezer. Beads extracted from each region are processed in separate reactions for the downstream library construction. Beads are washed sequentially with TE/SDS buffer (10 mM Tris-HCl, 1 mM EDTA, 0.5% SDS), TE/TW buffer, and nuclease-free water. cDNA amplification is performed in 50 µL PCR solution (1X Hifi Hot Start Ready mix (Kapa Biosystems, cat# KK2601), 1 µM SMRTpcr primer, with 14 amplification cycles (95° C. 3 min, 4 cycles of (98° C. 20 s, 65° C. 45 s, 72° C. 3 min), 10 cycles of (98° C. 20 s, 67° C. 20 s, 72° C. 3 min), 72° C. 5 min) on a thermocycler. PCR product from each piece is pooled and purified using SPRI paramagnetic bead (Beckman, cat# A63881) with a bead-to-sample volume ratio of 0.6:1. Purified cDNAs are then tagmented and amplified using the Nextera kit for in vitro transposition (Illumina, FC-131-1024). 0.8 ng cDNA is used as input per reaction. A unique i7 index primer is used to barcode the libraries obtained from each piece of the device. The i5 index primer is replaced by a universal P5 primer for the selective amplification of 5′ end of cDNA (corresponding to the 3′ end of RNA). Two rounds of SPRI paramagnetic bead-based purification with a bead-to-sample volume ratio of 0.6:1 and 1:1 are performed sequentially on the Nextera PCR product to obtain sequencing-ready libraries. The resulting single-cell RNA-Seq libraries are pooled and 20% PhiX library (Illumina, FC-131-1024) is spiked-in before sequencing on an Illumina NextSeq 500 with a 26-cycle read 1,58 cycle read 2, and8 cycle index read. A custom sequencing primer is used for read 1.

Automated reagent delivery system. An automated reagent delivery and scanning system is designed for automated optical decoding. In this system, fixed positive pressure (~1 psi) stabilized by a pressure regulator (SMC Pneumatics, cat# AW20-N02-Z-A) is used to drive fluid flow. The microwell device is constantly pressurized during incubation steps, which prevents evaporation and bubble formation. Two 10-channel rotary selector valves (IDEX Health & Science, cat# MLP778-605) are connected in parallel to toggle between 14 reagent channels. A three-way solenoid valve (Cole-Parmer, cat# EW-01540-11), located at the downstream of the microwell device, is used as an on/off switch for reagent flow. The multi-channel selector valves are controlled by a USB digital I/O device (National Instruments, cat# SCB-68A). The three-way solenoid valve is controlled by the same USB digital I/O device, but through a homemade transistor-switch circuit. The system is controlled by an imaging software (Nikon, NIS-Elements).

Bead optical decoding analysis. Eight cycles of probe hybridizations (A to H) are used for cell barcode optical decoding. For each cycle, the device is imaged in the bright-field, Cy3 and Cy5 emission channels. Beads are first identified in the bright-field image by the ImageJ Particle Analyzer plugin, and the positions of the beads in the bright-field image are recorded. Then the average fluorescence intensities of each bead in the Cy3 and Cy5 images are measured. Beads identified in cycles B to H are mapped to the nearest bead in cycle A. Thus, a probe hybridization matrix is obtained with n beads x 16 intensity values (8 for Cy3 and 8 for Cy5). To call cell barcodes from the imaging data, two methods are tested:

Cycle-by-cycle. In the cycle-by-cycle method, for each cycle and each fluorescent channel; Get N log transformed average intensity values; Compute an intensity histogram using 50 bins; Determine the median intensity value M, and identify the highest bin with intensity values smaller than M as B₁ and the highest bin with intensity values greater than M as B₂; Identify the lowest bin B₃ with intensity values between B₁ and B₂; Get the medium intensity value I of bin B₃, then assign 0 to intensity values smallerthan I and assign 1 to intensity values greater than I. Refer to the binary code table. If the code assigned is in the table, then return the corresponding cell identifying optical barcode sequence.

Bead-by-bead. In the bead-by-bead method, for each bead and each fluorescence channel; Get eight average fluorescence intensity values x₁,x₂,...,x₈; Let y₁, y₂,...,y₈ be the sorted values; Let fₙ = (yₙ₊₁ - yₙ)/yₙ, n = 1,2,...,7 be the relative intensity fold change between neighbor sorted values; Determine the largest fold change N = argmax(fₙ), then assign 0 to values to y₁,y₂,...,y_{N} and assign 1 to n values y_{N+1},y_{N+2},...,y₈; Refer to the binary code table. If the code assigned is in the table, then return the corresponding cell barcode sequence; Otherwise, remove f_{N} from list {fn} and repeat the process using the next largest fold change until a corresponding cell barcode sequence is returned or the list {fn} is empty.

### Example 6

### Accuracy of Linking Imaging and Sequencing Data Using RNA Capture Beads Containing Cell Identifying Optical Barcodes

An experiment is performed to demonstrate using RNA capture beads containing cell identifying optical barcodes to link single cell phenotypic image and nucleic acid sequence data, in terms of throughput, molecular capture efficiency, and accuracy of linking imaging and sequencing data.

This experiment is performed with mixed human (U87) and mouse (3T3) cells labeled with two differently colored live staining dyes. Mixed cells are loaded into the microwells at a relatively high density and 9,061 transcriptional profiles are obtained from a single experiment. At saturating sequencing depth, on average 10,245 RNA transcripts are detected from 3,548 genes per cell (FIGS. 18A, 18B). To evaluate the linking accuracy, the species of each cell is identified from the color of the fluorescent label and from the species-specific alignment rate in RNA-seq (a cell with >90% of reads aligning to the transcriptome of a given species is considered species-specific), and the consistency of the two cell species calls is examined. In the 4,145 scRNA-seq profiles that are successfully linked with imaging data, a class-balanced linking accuracy of 99.2% (0.8% error rate) is obtained, with 98.8% of human cells and 99.6% of mouse cells agreeing with the species calls from two-color imaging (FIG. 18C). In addition, multiplets are confidently removed by manually identifying mixed-species and single-species multiplets from the two-color cell images. By comparing image-based and sequencing-based mixed-species multiplets, a multiplet detection sensitivity of 68.8% and a specificity of 97.0% is obtained. A large portion of transcriptional profiles with low purity are removed (FIG. 18D). Since high linking accuracy is confirmed, it is suspected that the mixed-species multiplets detected by sequencing but not imaging are because of the imperfections in scRNA-seq data, which is serving as the ground truth.

### Methods

Cell culture. Human U87 and mouse 3T3 cells are cultured in Dulbecco’s modified eagle medium (DMEM, Life Technologies, cat# 11965118) supplemented with 10% fetal bovine serum (FBS, Life Technologies, cat# 16000044) at 37° C. and 5% carbon dioxide.

Human and mouse cells mixed experiment. Human U87 cells are stained with Calcein AM (ThermoFisher Scientific, cat# C3100MP) and mouse 3T3 cells are stained with Calcein red-orange (ThermoFisher Scientific, cat# C34851) in culture medium at 37° C. for 10 minutes. The stained cells are then dissociated into single cell suspension by 0.25% Trypsin-EDTA (Life Technologies, cat# 25200-072) and re-suspended in TBS buffer. The U87 and 3T3 cells are mixed at 1:1 ratio with a final total cell concentration 1000 cells/µl. The mixed cell suspension is processed and sequenced and images and sequencing data are processed as described above in Example 5.

Imaging based multiplets identification. Two-color live staining fluorescence images are merged with Calcein AM signal in green and Calcein red-orange signal in magenta. Each well is manually examined within the smallest bounding square. Wells with mixed-species cells are determined as having at least one green object and one magenta object; wells with a single cell are determined as having only one green object or one magenta object.

Sub-sampling analysis. To analyze the saturation behavior and sensitivity of scRNA-seq data (FIG. 18A), the aligned reads are randomly sub-sampled and re-processed with the scRNA-seq analysis using the procedure described herein above. Two statistics are then calculated, molecules per cell and genes per cell, based on the cells that are discovered from the total reads.

Accuracy of linking imaging and scRNA-seq data. The linking accuracy is defined as the concordance between the scRNA-seq and imaging-based species calling for cell barcodes associated with a single species. In scRNA-seq data, cells with >90% of reads aligning uniquely to a given species are considered to correspond to a single species. In the imaging data, the imaging-based species call is determined based on cell live staining colors. Cells with Calcein AM intensity > 724 are called as imaging-based human cells; Cells with Calcein red-orange intensity > 2,048 are called as imaging-based mouse cells. Intensity thresholds are determined as the intensity of the shortest bin between the two mean values of the bimodal Gaussian distribution of intensity values.

### Example 7

### Integration of Single-Cell RNA-Seq and Cell Phenotype Image Analysis in Human Glioblastoma Samples

To demonstrate collection of paired optical and transcriptional phenotypes from human tissue samples using the cell identifying optical barcodes described herein, an experiment is performed on cells dissociated from a human glioblastoma (GBM) surgical sample and labeled with calcein AM, a fluorgenic dye that reports esterase activity. 1,954 scRNA-seq profiles are obtained and 1,110 of them linked to live cell images. Cell multiplets are removed based on imaging analysis. Calcein AM is commonly used as a live stain and, thus, outlier cells with low fluorescence intensity are also removed. Malignantly transformed GBM cells often resemble non-neoplastic neural cell types in the adult brain, and thus simple marker-based analysis is insufficient to confirm malignant status. To address this, a large population of cells is identified with amplification of chromosome 7 and loss of chromosome 10, two commonly co-occurring aneuploidies that are pervasive in GBM, based on the gene expression. A low-dimensional representation is then computed of the data using single-cell hierarchical Poisson factorization (scHPF) to identify key gene signatures that define the population and visualized their distributions across cells using Uniform Manifold Approximation and Projection (UMAP). All of the major cell types are recovered that have been previously reported from scRNA-seq of GBM including myeloid cells, endothelial cells, pericytes, malignant-transformed astrocyte-like cells, mesenchymal-like cells, oligodendrocyte-progenitor-like/neuroblast-progenitor-like cells (OPC/NPC) and cycling cells (FIGS. 19A, 19B). Sixteen imaging features are measured from cell images and those features grouped into three categories of cell size, shape and calcein AM intensity using unsupervised hierarchical clustering (FIG. 19C) to create three imaging-based meta-features. By linking the meta-features to scRNA-seq cell types, myeloid cells (clusters 7 and 12) are found to be relatively round and small with high esterase activity; endothelial cells are large and less round as expected, and have intermediate esterase activity; and pericytes have intermediate shape, size and intensity (FIG. 19D).

Identification of Relationships between Imaging Features and Lineage Identities of Malignantly Transformed GBM Cells. Malignant cells in GBM can resemble multiple neural lineages and exhibit a mesenchymal phenotype. Because malignant GBM cells are known to be highly plastic and undergo differentiation and de-differentiation, a diffusion map is used to visualize their lineage relationships. Malignant cells are selected based on aneuploidy as described above, the dimensionality of malignant cell gene expression is reduced by scHPF, and the factorized data are visualized with a diffusion map, which reveals two major branches. One branch consists of astrocyte-like cells and terminates with mesenchymal-like cells, while the other branch consists of OPC/NPC cells and cycling cells. This is consistent with previously published studies showing that astrocyte-like and mesenchymal glioma cells are significantly more quiescent than OPC-like glioma cells.

To explore how imaging features of malignant cells are related to the two major cellular lineages, it is asked whether unsupervised clustering of cellular imaging features would correspond to the two major lineages observed in scRNA-seq. Malignant cells are clustered by the three imaging meta-features described above using hierarchical clustering, and two major cellular imaging clusters are identified. By plotting two imaging clusters on the diffusion map embedding of the malignant cells, it is found that cells with round shape, low intensity and small size (imaging cluster 0) are enriched in the OPC/NPC-cycling branch, and cells with rough shape, high intensity and large size (imaging cluster 1) are enriched in the astrocyte-mesenchymal branch (FIG. 20D). This finding is further supported by differential expression analysis comparing expression profiles of cells in the two imaging clusters. As expected, markers of OPC/NPCs (MAP2, OLIG1, DLL3) and cycling cells (CDK6) are significantly enriched (FDR<0.05, Mann-Whitney U-test) in imaging cluster 0, while markers of astrocyte-like cells (APOE, GFAP, GJA1, AQP4, ALDOC) and mesenchymal cells (CHl3L1, CD44, CHl3L2, CCL2) are significantly enriched (FDR<0.05, Mann-Whitney U-test) in imaging cluster 1. Therefore, there is a clear correspondence between the major gene expression and basic imaging features for the malignantly transformed cells in this tumor.

### Methods

GBM tissue processing. A single-cell suspension is obtained from excess material collected during surgical resection of a WHO Grade IV GBM. The patient is anonymous and the specimen is de-identified. The tissue is mechanically dissociated following a 30-minute incubation with papain at 37° C. in Hank’s balanced salt solution. Cells are re-suspended in TBS after centrifugation at 100xg followed by selective lysis of red blood cells with ammonium chloride for 15 minutes at room temperature. Finally, cells are washed with TBS and quantified using a Countess (ThermoFisher). Cells are stained with Calcein AM (ThermoFisher Scientific, cat# C3100MP). The GBM cell suspension is processed and sequenced using RNA capture beads containing the cell identifying optical barcodes and imaging and sequencing data are processed as described herein in Examples 5-7. Multiplets are removed based on manual examination of each well within the smallest bounding square of the Calcein AM fluorescence image. The dead cells are identified based on the Calcein AM fluorescence intensity. A Gaussian distribution is fitted to the fluorescent intensity histogram, a threshold of lower5 percentile is set, and cells with intensity lower than the threshold are removed.

Live cell imaging analysis. Images are analyzed using ImageJ software. To identify microwells with cells, microwell outlines are identified as objects from the bright-field image using a local threshold, and then average fluorescence intensities of microwells in the live staining images are measured. Average intensity values follow a bimodal distribution, with the higher intensity population corresponding to microwells that contain cells. To extract cell optical phenotypes, only microwells with cells are selected and each cell is analyzed individually within the smallest bounding square of the corresponding microwell. The cell is identified in the live staining fluorescence image using the auto threshold and particle analyzer. Microwells with multiple cells identified by the software are excluded. Sixteen imaging features are measured for each cell in the fluorescence image: area, mean intensity, standard deviation of intensity, minimum intensity, maximum intensity, median intensity, perimeter, width, height, major axis, minor axis, circularity, Feret’s diameter, minimum Feret’s diameter, roundness, and solidity.

Analysis of scRNA-seq with optically barcoded beads. To analyze the scRNA-seq data collected using beads containing cell identifying optical barcode sequences, the cell-identifying optical barcode and UMI from Read 1 is first extracted based on the designed oligonucleotide sequence, NN(8-nt Cell Barcode S)NN(8-nt Cell Barcode Q)NNNN. The 192 8-nt cell barcode sequences have a Hamming distance of at least three for all sequence pairs. Therefore, one substitution error is corrected in the cell barcode sequences. Only reads with a complete cell barcode are retained. Next, the reads are aligned from Read 2 to a merged human/mouse genome (GRCh38 for human and GRCm38 for mouse) with merged GENCODE transcriptome annotations (GENCODE v.24 for both species) using STAR v.2.7.0 aligner after removal of 3′ poly(A) tails (indicated by tracts of >7 A’s) and fragments with fewer than 24 nucleotides after poly(A) tail removal. Only reads that uniquely mapped to exons on the annotated strand are included for the downstream analysis. Reads with the same cell barcode, UMI (after one substitution error correction) and gene mapping are considered to originate from the same cDNA molecule and collapsed. Finally, this information is used to generate a molecular count matrix.

Optically barcoded beads for linking cell imaging and sequencing data. To link the cell identifying optical barcodes identified from imaging to cell imaging phenotypes, bright-field images of the microwell device obtained during optical decoding are mapped to images of the live cell imaging based on the upper-left and the bottom right microwells. Cells are then registered to the nearest mapped bead within a microwell radius. To link cell imaging phenotypes to expression profiles, only cell barcodes with registered cells are considered, and then the exact and unique mapping of the cell identifying optical barcodes from imaging and sequencing is found.

Single cell hierarchical Poisson factorization (scHPF) analysis. To reduce the dimensionality of scRNA-seq results, the gene count matrix is factorized using the scHPF with default parameters and K = 13. One of the factors contains several heat shock with high gene scores (among the top 50 genes), likely indicating dissociation artifacts in certain cells. This factor is removed in all downstream analysis.

Malignant cell identification. The cell aneuploidy analysis was performed based on the scHPF model as described previously. To compute the scHPF-imputed expression matrix, the gene and cell weight matrix (expectation matrix of variable θ and β) is multiplied in the scHPF model and then the result matrix log-transformed as log₂(expected counts/10000 + 1) . The average gene expression on each somatic chromosome is calculated using the scHPF-imputed count matrix as previously described. A malignancy score is defined as the difference between the average expression of Chr. 7 genes to that of Chr. 10 genes, < log₂(Chr. 7 Expression) > - < log₂(Chr. 10 Expression) > . A double Gaussian distribution is fitted to the malignancy score and the score of the shortest bin between two mean intensities is used as the threshold that separates the malignant and non-malignant cell populations. The difference of chromosome average expression between malignant and non-malignant cells is computed as the expression subtracted by the average expression of non-malignant cells.

scRNA-seq clustering and visualization. To visualize the scHPF model (FIG. 19A), a UMAP embedding is generated using the Pearson correlation distance matrix computed from the cell score matrix. To cluster the scRNA-seq profiles, the Phenograph implementation of Louvain community detection is used, with the Pearson correlation matrix and k=50 to construct a k-nearest neighbors graph.

Cell optical phenotypes clustering. To reduce the dimensionality of the cellular imaging features, 16 cell imaging features are z-normalized and hierarchically clustered using the ‘linkage’ method in the python module ‘SciPy’ with correlation distance. The dendrogram in FIG. 19C is cut as k=3 to form three clusters of imaging features, corresponding to cell size, shape, and esterase activity. The values of meta-features are calculated as an average of the imaging features within each cluster. To cluster the malignant cells based on their optical phenotypes, imaging meta-features are hierarchically clustered using the ‘linkage’ method in the python module ‘SciPy’ with correlation distance.

Diffusion map embedding of malignantly transformed GBM cells. The molecular count matrix for malignantly transformed GBM cells (identified by aneuploidy analysis as described above) is factorized using scHPF with default parameters and K=15. Prior to further analysis, one of the 15 factors is removed, which exhibits high scores for heat shock response genes, because it likely represents a dissociation artifact in a subset of cells. Diffusion components are then computed with the DMAPS Python library. A Pearson correlation distance matrix computed from the scHPF cell score matrix is used as input with a kernel bandwidth of 0.5. The first two diffusion components are plotted in FIG. 19D.

scRNA-seq differential expression. The Mann-Whitney U-test is used for differential expression analysis. For pairwise comparison of two groups of cells, the group with more cells is randomly sub-sampled to the same cell number as the group with fewer cells. Next, the detected molecules from the group with a higher average number of molecules detected per cell are randomly sub-sampled so that the two groups had the same average number of molecules detected per cell. The resulting sub-sampled matrices are then normalized using a random pooling method as implemented in the scran R package. Finally, the resulting normalized matrices are subjected to gene-by-gene differential expression testing using the Mann-Whitney U-test using the ‘mannwhitneyu’ function in the Python package SciPy. The resulting p-values are corrected using the Benjamini-Hochberg method as implemented in the ‘multipletests’ function in the Python package statsmodels.

### REFERENCES

1. Macosko EZ, Basu A, Satija R, Nemesh J, Shekhar K, Goldman M, Tirosh I, Bialas AR, Kamitaki N, Martersteck EM, et al: Highly Parallel Genome-wide Expression Profiling of Individual Cells Using Nanoliter Droplets. Cell 2015, 161:1202-1214.
2. Klein AM, Mazutis L, Akartuna I, Tallapragada N, Veres A, Li V, Peshkin L, Weitz DA, Kirschner MW: Droplet barcoding for single-cell transcriptomics applied to embryonic stem cells. Cell 2015, 161:1187-1201.
3. Bose S, Wan Z, Carr A, Rizvi AH, Vieira G, Pe’er D, Sims PA: Scalable microfluidics for single-cell RNA printing and sequencing. Genome Biol 2015, 16:120.
4. Rotem A, Ram O, Shoresh N, Sperling RA, Schnall-Levin M, Zhang H, Basu A, Bernstein BE, Weitz DA: High-Throughput Single-Cell Labeling (Hi-SCL) for RNA-Seq Using Drop-Based Microfluidics. PLoS One 2015, 10:e0116328.
5. Fan HC, Fu GK, Fodor SP: Expression profiling. Combinatorial labeling of single cells for gene expression cytometry. Science 2015, 347:1258367.
6. Shalek AK, Satija R, Adiconis X, Gertner RS, Gaublomme JT, Raychowdhury R, Schwartz S, Yosef N, Malboeuf C, Lu D, et al: Single-cell transcriptomics reveals bimodality in expression and splicing in immune cells. Nature 2013, 498:236-240.
7. Lane K, Van Valen D, DeFelice MM, Macklin DN, Kudo T, Jaimovich A, Carr A, Meyer T, Pe’er D, Boutet SC, Covert MW: Measuring Signaling and RNA-Seq in the Same Cell Links Gene Expression to Dynamic Patterns of NF-kappaB Activation. Cell Syst 2017, 4:458-469 e455.
8. Goldstein LD, Chen YJ, Dunne J, Mir A, Hubschle H, Guillory J, Yuan W, Zhang J, Stinson J, Jaiswal B, et al: Massively parallel nanowell-based single-cell gene expression profiling. BMC Genomics 2017, 18:519.
9. Yuan J, Sims PA: An Automated Microwell Platform for Large-Scale Single Cell RNA-Seq. Sci Rep 2016, 6:33883.
10. Gierahn TM, Wadsworth MH, 2nd, Hughes TK, Bryson BD, Butler A, Satija R, Fortune S, Love JC, Shalek AK: Seq-Well: portable, low-cost RNA sequencing of single cells at high throughput. Nat Methods 2017, 14:395-398.
11. Love JC, Ronan JL, Grotenbreg GM, van der Veen AG, Ploegh HL: A microengraving method for rapid selection of single cells producing antigen-specific antibodies. Nature Biotechnology 2006, 24:703-707.
12. Sims CE, Allbritton NL: Analysis of single mammalian cells on-chip. Lab on a Chip 2007, 7:423-440.

1. An automated system for associating single cell imaging with unique optical barcode readout, and preparation of RNA libraries, the system comprising:
an instrument assembly comprising a fluidics subsystem, a thermal subsystem, and an imaging subsystem, wherein the imaging subsystem comprises a stage configured for holding a microwell array;
a control subsystem coupled to the instrument assembly, the control subsystem comprising at least one processor and memory, the control subsystem configured for performing operations comprising:
flowing, using the fluidics subsystem, a plurality of cells onto the microwell array, wherein a subset of the cells reside as single cells in the microwells;
obtaining, for each position of a plurality of positions in the microwell array, one or more first images of the cell at the position using the imaging subsystem;
flowing, using the fluidics subsystem, a plurality of microbeads having a cell identifying optical barcode sequence and an RNA binding sequence onto the microwell array, wherein a subset of the beads reside as a single cell-bead pair in the microwells;
flowing, using the fluidics subsystem, a cell lysis buffer and one or more reagents for RNA library preparation onto the microwell array;
flowing, using the fluidics subsystem, a first of N pools of a plurality of optical hybridization probes onto the microwell array and hybridizing the probes to the beads located therein having a complementary nucleotide sequence in the cell identifying optical barcode sequence;
obtaining, for each position of the plurality of positions, one or more second images to quantify a fluorescent intensity at the position using the imaging subsystem, each of the one or more second images used to create a binary code depicting a match or a lack of a match between at least one of the optical hybridization probes and the cell identifying optical barcodes;
repeating the flowing and hybridizing step and obtaining of the one or more second images step for each of the N pools of probes; and
determining, by mapping the binary code for each of the N pools of probes to the cell identifying barcode sequence, for each position of the plurality of positions, the cell identifying optical barcode for the position and storing a data association between the cell identifying optical barcode for the position and the first image at the position.

2. The system of claim 1, the operations comprising:
imaging, using the imaging subsystem, the microwell array and performing image analysis to monitor cell lysis for completion within the microwells.

3. The system of claim 1, wherein the one or more reagents for RNA library sample preparation include a reverse transcription mix, and the operations comprising:
flowing, using the fluidics subsystem, reverse transcription mix onto the microwell array after determining completion of cell lysis based on performing image analysis.

4. The system of claim 1, the operations comprising:
determining, for each position of the plurality of positions, a number of cells depicted in a microwell corresponding to the position using the first image of the position.

5. The system of claim 1, the operations comprising: recovering the microbeads.

6. The system of claim 1, the operations comprising:
receiving, for each cell identifying optical barcode, nucleic acid sequencing data; and
storing a data association between the nucleic acid sequencing data, the cell identifying optical barcode, and the first image associated with the cell identifying optical barcode.

7. The system of claim 1, comprising a microwell array.

8. The system of claim 1, wherein the thermal subsystem is in thermal connection with the stage holding the microwell array, and wherein the operations comprise controlling the thermal subsystem to apply heat to the microwell array.

9. The system of claim 1, wherein the fluidics subsystem comprises a flow rate unit, a flow control unit, one or more valving units, and one or more pressurized reagent reservoirs, and wherein the operations comprise controlling the flow control unit and controlling valve switching.

10. An automated method for associating single cell imaging data with RNA transcriptomics, the method comprising:
initializing a system, the system comprising:
an instrument assembly comprising a fluidics subsystem, a thermal subsystem, and an imaging subsystem, wherein the imaging subsystem comprises a stage configured for holding a microwell array;
a control subsystem coupled to the instrument assembly, the control subsystem comprising at least one processor and memory; and
using the control subsystem for performing operations comprising:
flowing, using the fluidics subsystem, a plurality of cells onto the microwell array, wherein a subset of the cells reside as single cells in the microwells;
obtaining, for each position of a plurality of positions in a microwell array, one or more first images at the position using the imaging subsystem;
flowing, using the fluidics subsystem, a plurality of microbeads having a cell identifying optical barcode sequence and an RNA binding sequence onto the microwell array, wherein a subset of the beads reside as a single cell-bead pair in the microwells;
flowing, using the fluidics subsystem, a cell lysis buffer and one or more reagents for RNA library preparation onto the microwell array;
flowing, using the fluidics subsystem, a first of N pools of a plurality of optical hybridization probes onto the microwell array and hybridizing the probes to the beads located therein having a complementary nucleotide sequence in the cell identifying optical barcode sequence;
obtaining, for each position of the plurality of positions, one or more second images to quantify a fluorescent intensity at the position using the imaging subsystem, each of the one or more second images used to create a binary code depicting a match or a lack of a match between at least one of the optical hybridization probes and the cell identifying optical barcodes;
repeating the flowing and hybridizing step and obtaining of the one or more second images step for each of the N pools of probes;
determining, by mapping the binary code for each of the N pools of probes to the cell identifying barcode sequence, for each position of the plurality of positions, the cell identifying optical barcode for the position and storing a data association between the cell identifying optical barcode for the position and the first image at the position; and
storing, for each position of the plurality of positions, after receiving nucleic acid sequencing data for each cell identifying optical barcode, a data association between the nucleic acid sequencing data, the cell identifying optical barcode, and the first image associated with the cell identifying optical barcode
wherein the single cell imaging data is thereby associated with the RNA transcriptome for that cell.

11. The method of claim 10, comprising:
imaging, using the imaging subsystem, the microwell array and performing image analysis to monitor cell lysis for completion within the microwells.

12. The method of claim 11, wherein the one or more reagents for RNA library preparation includes a reverse transcription mix, and comprising:
flowing, using the fluidics subsystem, reverse transcription mix onto the microwell array after determining completion of cell lysis based on performing image analysis.

13. The method of claim 10, comprising:
determining, for each position of the plurality of positions, a number of cells depicted in a microwell corresponding to the position using the first image of the position.

14. The method of claim 10, comprising recovering the microbeads.

15. The method of claim 10, comprising controlling a thermal subsystem to apply heat to the microwell array.

16. The method of claim 10, wherein the fluidics subsystem comprises a flow rate unit, a flow control unit, one or more valving units, and one or more pressurized reagent reservoirs, and wherein the method comprises controlling the flow control unit and controlling valve switching.

17. The method of claim 10, wherein the obtaining the one or more first images at the position using an imaging subsystem, further comprises:
measuring one or more of a cell optical phenotypic feature; and
generating a representation of the relationship between the one or more cell optical phenotypic features and the nucleic acid sequencing data associated with each of the first images,
wherein a correlation between the single cell phenotypic features and the associated sequencing data identifies a correspondence between single cell optical phenotypes and cell type, lineage, or clone based on transcriptomics of that single cell.

18. The method of claim 10, wherein the cell optical phenotypic feature comprises one or more of area, mean intensity, standard deviation of intensity, minimum intensity, maximum intensity, median intensity, perimeter, width, height, major axis, minor axis, circularity, Feret’s diameter, minimum Feret’s diameter, roundness, or solidity.

19-27. (canceled)

28. An automated system for associating single cell imaging with unique optical barcode readout, and preparation of sequencing libraries, the system comprising:
an instrument assembly comprising a fluidics subsystem, a thermal subsystem, and an imaging subsystem, wherein the imaging subsystem comprises a stage configured for holding a microwell array;
a control subsystem coupled to the instrument assembly, the control subsystem comprising at least one processor and memory, the control subsystem configured for performing operations comprising:
flowing, using the fluidics subsystem, a plurality of cells onto the microwell array, wherein a subset of the cells reside as single cells in the microwells;
obtaining, for each position of a plurality of positions in the microwell array, one or more first images of the cell at the position using the imaging subsystem;
flowing, using the fluidics subsystem, a plurality of microbeads having a cell identifying optical barcode sequence and a primer sequence to capture cellular nucleic acid onto the microwell array, wherein a subset of the beads reside as a single cell-bead pair in the microwells;
flowing, using the fluidics subsystem, a cell lysis buffer and one or more reagents for sequencing library preparation onto the microwell array;
flowing, using the fluidics subsystem, a first of N pools of a plurality of optical hybridization probes onto the microwell array and hybridizing the probes to the beads located therein having a complementary nucleotide sequence in the cell identifying optical barcode sequence;
obtaining, for each position of the plurality of positions, one or more second images to quantify a fluorescent intensity at the position using the imaging subsystem, each of the one or more second images used to create a binary code depicting a match or a lack of a match between at least one of the optical hybridization probes and the cell identifying optical barcodes;
repeating the flowing and hybridizing step and obtaining of the one or more second images step for each of the N pools of probes; and
determining, by mapping the binary code for each of the N pools of probes to the cell identifying barcode sequence, for each position of the plurality of positions, the cell identifying optical barcode for the position and storing a data association between the cell identifying optical barcode for the position and the first image at the position.

29. (canceled)

30. The system of claim 28, the operations comprising:
imaging, using the imaging subsystem, the microwell array and performing image analysis to monitor cell lysis for completion within the microwells.

31-53. (canceled)
